SEQUENCE LISTING (4 sequences)

SEQ ID NO: 1 - DNA, 533 bp, Homo sapiens
tgctcagttcatccctagaggcagctgctccaggaacagaggtgccatgcagccccgggtactccttgttgttgccctcctggcgctcctggcctctgcccgagcttcagaggccgaggatgcctcccttctcagcttcatgcagggttacatgaagcacgccaccaagaccgccaaggatgcactgagcagcgtgcaggagtcccaggtggcccagcaggccaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatg

SEQ ID NO: 2 - DNA, 3964 bp, Homo sapiens
ctactccaggctgtgttcagggcttggggctggtggagggaggggcctgaaattccagtgtgaaaggctgagatgggcccgaggcccctggcctatgtccaagccatttcccctctcaccagcctctccctggggagccagtcagctaggaaggaatgagggctccccaggcccacccccagttcctgagctcatctgggctgcagggctggcgggacagcagcgtggactcagtctcctagggatttcccaactctcccgcccgcttgctgcatctggacaccctgcctcaggccctcatctccactggtcagcaggtgacctttgcccagcgccctgggtcctcagtgcctgctgccctggagatgatataaaacaggtcagaaccctcctgcctgtctgctcagttcatccctagaggcagctgctccaggtaatgccctctggggaggggaaagaggaggggaggaggatgaagaggggcaagaggagctccctgcccagcccagccagcaagcctggagaagcacttgctagagctaaggaagcctcggagctggacgggtgccccccacccctcatcataacctgaagaacatggaggcccgggaggggtgtcacttgcccaaagctacacagggggtggggctggaagtggctccaagtgcaggttcccccctcattcttcaggcttagggctggaggaagccttagacagcccagtcctaccccagacagggaaactgaggcctggagagggccagaaatcacccaaagacacacagcatgttggctggactggacggagatcagtccagaccgcaggtgccttgatgttcagtctggtgggttttctgctccatcccacccacctccctttgggcctcgatccctcgcccctcaccagtcccccttctgagagcccgtattagcagggagccggcccctactccttctggcagacccagctaaggttctaccttaggggccacgccacctccccagggaggggtccagaggcatggggacctggggtgcccctcacaggacacttccttgcaggaacagaggtgccatgcagccccgggtactccttgttgttgccctcctggcgctcctggcctctgcccgtaagcacttggtgggactgggctgggggcagggtggaggcaacttggggatcccagtcccaatgggtggtcaagcaggagcccagggctcgtccagaggccgatccaccccactcagccctgctctttcctcaggagcttcagaggccgaggatgcctcccttctcagcttcatgcagggttacatgaagcacgccaccaagaccgccaaggatgcactgagcagcgtgcaggagtcccaggtggcccagcaggccaggtacacccgctggcctccctccccatcccccctgccagctgcctccattcccacccgcccctgccctggtgagatcccaacaatggaatggaggtgctccagcctcccctgggcctgtgcctcttcagcctcctctttcctcacagggcctttgtcaggctgctgcgggagagatgacagagttgagactgcattcctcccaggtccctcctttctccccggagcagtcctagggcgtgccgttttagccctcatttccattttcctttcctttccctttctttctctttctatttctttctttctttctttctttctttctttctttctttctttctttctttctttctttctttctttcctttctttctttcctttctttctttcctttctttctttctttcctttctttctctttctttctttctttcctttttctttctttccctctcttcctttctctctttctttcttcttcttttttttttaatggagtctccctctgtcacctaggctggagtgcagtggtgccatctcggctcactgcaacctccgtctcccgggttcaacccattctcctgcctcagcctcccaagtagctgggattacaggcacgcgccaccacacccagctaatttttgtatttttagcagagatggggtttcaccatgttggccaggttggtcttgaattcctgacctcaggggatcctcctgcctcggcctcccaaagtgctgggattacaggcatgagccactgcgcctggccccattttccttttctgaaggtctggctagagcagtggtcctcagcctttttggcaccagggaccagttttgtggtggacaatttttccatgggccagcggggatggttttgggatgaagctgttccacctcagatcatcaggcattagattctcataaggagccctccacctagatccctggcatgtgcagttcacaatagggttcacactcctatgagaatgtaaggccacttgatctgacaggaggcggagctcaggcggtattgctcactcacccaccactcacttcgtgctgtgcagcccggctcctaacagtccatggaccagtacctatctatgacttgggggttggggacccctgggctaggggtttgccttgggaggccccacctgacccaattcaagcccgtgagtgcttctgctttgttctaagacctggggccagtgtgagcagaagtgtgtccttcctctcccatcctgcccctgcccatcagtactctcctctcccctactcccttctccacctcaccctgactggcattagctggcatagcagaggtgttcataaacattcttagtccccagaaccggctttggggtaggtgttattttctcactttgcagatgagaaaattgaggctcagagcgattaggtgacctgccccagatcacacaactaatcaatcctccaatgactttccaaatgagaggctgcctccctctgtcctaccctgctcagagccaccaggttgtgcaactccaggcggtgctgtttgcacagaaaacaatgacagccttgacctttcacatctccccaccctgtcactttgtgcctcaggcccaggggcataaacatctgaggtgacctggagatggcagggtttgacttgtgctggggttcctgcaaggatatctcttctcccagggtggcagctgtgggggattcctgcctgaggtctcagggctgtcgtccagtgaagttgagagggtggtgtggtcctgactggtgtcgtccagtggggacatgggtgtgggtcccatggttgcctacagaggagttctcatgccctgctctgttgcttcccctgactgatttaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgcgagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatgagtgggccgtcgcaagtgtgccatctgtgtctgggcatgggaaagggccgaggctgttctgtgggtgggcactggacagactccaggtcaggcaggcatggaggccagcgctctatccaccttctggtagctgggcagtctctgggcctcagtttcttcatctctaaggtaggaatcaccctccgtaccctgccttccttgacagctttgtgcggaaggtcaaacaggacaataagtttgctgatactttgataaactgttaggtgctgcacaacatgacttgagtgtgtgccccatgccagccactatgcctggcacttaagttgtcatcagagttgagactgtgtgtgtttactcaaaactgtggagctgacctcccctatccaggccccctagccct

SEQ ID NO: 3 - DNA, 20 bp, Artificial sequence
  Synthetic oligonucleotide
agcttcttgtccagctttat

SEQ ID NO: 4 - DNA, 3958 bp, Homo sapiens
ctactccaggctgtgttcagggcttggggctggtggagggaggggcctgaaattccagtgtgaaaggctgagatgggcccgaggcccctggcctatgtccaagccatttcccctctcaccagcctctccctggggagccagtcagctaggaaggaatgagggctccccaggcccacccccagttcctgagctcatctgggctgcagggctggcgggacagcagcgtggactcagtctcctagggatttcccaactctcccgcccgcttgctgcatctggacaccctgcctcaggccctcatctccactggtcagcaggtgacctttgcccagcgccctgggtcctcagtgcctgctgccctggagatgatataaaacaggtcagaaccctcctgcctgtctgctcagttcatccctagaggcagctgctccaggtaatgccctctggggaggggaaagaggaggggaggaggatgaagaggggcaagaggagctccctgcccagcccagccagcaagcctggagaagcacttgctagagctaaggaagcctcggagctggacgggtgccccccacccctcatcataacctgaagaacatggaggcccgggaggggtgtcacttgcccaaagctacatagggggtggggctggaagtggctccaagtgcaggttcccccctcattcttcaggcttagggctggaggaagccttagacagcccagtcctaccccagacagggaaactgaggcctggagagggccagaaatcacccaaagacacacagcatgttggctggactggacggagatcagtccagaccgcaggtgccttgatgttcagtctggtgggttttctgctccatcccacccacctccctttgggcctcgatccctcgcccctcaccagtcccccttctgagagcccgtattagcagggagccggcccctactccttctggcagacccagctaaggttctaccttaggggccacgccacctccccagggaggggtccagaggcatggggacctggggtgcccctcacaggacacttccttgcaggaacagaggtgccatgcagccccgggtactccttgttgttgccctcctggcgctcctggcctctgcccgtaagcacttggtgggactgggctgggggcagggtggaggcaacttggggatcccagtcccaatgggtggtcaagcaggagcccagggctcgtccataggccgatccaccccactcagccctgctctttcctcaggagcttcagaggccgaggatgcctcccttctcagcttcatgcagggctacatgaagcacgccaccaagaccgccaaggatgcactgagcagcgtgcaggagtcccaggtggcccagcaggccaggtacacccgctggcctccctccccatcccccctgccagctgcctccattcccacccacccctgccctggtgagatcccaacaatggaatggaggtgctccagcctcccctgggcctgtgcctcttcagcctcctctttcctcacagggcctttgtcaggctgctgcgggagagatgacagagttgagactgcattcctcccaggtccctcctttctccccagagcagtcctagggcgcgccgttttagccctcatttccattttcctttcctttccctttctttccctttctatttctttctttctttctttctttctttctttctttctttctttctttctttctttctttctttctttcctttctttctttcttttcttctttctttctttcctttctttctctttctttctttctttctttcctttttctttctttccctctcttcctttctctctttctttcttcttcttttttttttaatggagtctccctctgtcacccaggctggagtgcagtggtgccatctcggctcactgcaacctccgtctcccgggttcaacccattctcctgcctcagcctcccaagtagctgggattacaggcacgcgccaccacacccagctaatttttgtatttttagcagagatggggtttcaccatgttggccaggttggtcttgaattcctgacctcaggggatcctcctgcctcggcctcccaaagcgctgggattacaggcatgagccactgcgcctggccccattttccttttctgaaggtctggctagagcagtggtcctcagcctttttggcaccagggaccagttttgtggtggacaatttttccatgggccagcggggatggttttgggatgaagctgttccacctcagatcatcaggcattagattctcataaggagccctccacctagatccctggcatgtgcagttcacaacagggttcacactcctatgagaatgtaaggccacttgatctgacaggaggcggagctcaggcggtattgctcactcacccaccactcacttcgtgctgtgcagcccggctcctaacagtccatggaccagtacctatctatgacttgggggttggggacccctgggctaggggtttgccttgggaggccccacctgacctaattcaagcccgtgagtgcttctgctttgttctaagacctggggccagtgtgagcagaagtgtgtccttcctctcccatcctgcccctgcccatcagtactctcctctcccctactcccttctccacctcaccctgactggcattagctggcatagcagaggtgttcataaacattcttagtccccagaaccggctttggggtaggtgttattttctcactttgcagatgagaaaattgaggctcagagcgattaggtgacctgccccagatcacacaactaatcaatcctccaatgactttccaaatgagaggctgcctccctctgtcctaccctgctcagagccaccaggttgtgcaactccaggcggtgctgtttgcacagaaaacaatgacagccttgacctttcacatctccccaccctgtcactttgtgcctcaggcccaggggcataaacatctgaggtgacctggagatggcagggtttgacttgtgctggggttcctgcaaggatatctcttctcccagggtggcagctgtgggggattcctgcctgaggtctcagggctgtcgtccagtgaagttgagagggtggtgtggtcctgactggtgtcgtccagtggggacatgggtgtgggtcccatggttgcctacagaggagttctcatgccctgctctgttgcttcccctgactgatttaggggctgggtgaccgatggcttcagttccctgaaagactactggagcaccgttaaggacaagttctctgagttctgggatttggaccctgaggtcagaccaacttcagccgtggctgcctgagacctcaataccccaagtccacctgcctatccatcctgccagctccttgggtcctgcaatctccagggctgcccctgtaggttgcttaaaagggacagtattctcagtgctctcctaccccacctcatgcctggcccccctccaggcatgctggcctcccaataaagctggacaagaagctgctatgagtgggccgtcgcaagtgtgccatctgtgtctgggcatgggaaagggccgaggctgttctgtgggtgggcactggacagactccaggtcaggcaggcatggaggccagcgctctatccaccttctggtagctgggcagtctctgggcctcagtttcttcatctctaaggtaggaatcaccctccgtaccctgccttccttgacagctttgtgcggaaggtcaaacaggacaataagtttgctgatactttgataaactgttaggtgctgcacaacatgacttgagtgtgtgccccatgccagccactatgcctggcacttaagttgtcatcagagttgagactgtgtgtgtttactcaaaactgtggagctgacctcccctatccaggccacctagccct

(19) United States
(12) Patent Application Publication
(10) Pub. No.: US 2018/0245076 A1
(43) Pub. Date: Aug. 30, 2018

(54) MODULATION OF APOLIPOPROTEIN C-III (APOCIII) EXPRESSION IN LIPODYSTROPHY POPULATIONS

(71) Applicant: Ionis Pharmaceuticals, Inc., Carlsbad, CA (US)

(72) Inventor: Andres Digenio, Cambridge, MA (US)

(73) Assignee: Ionis Pharmaceuticals, Inc., Carlsbad, CA (US)

(21) Appl. No.: 15/553,946

(22) PCT Filed: Feb. 26, 2016
(86) PCT No.: PCT/US2016/019728

§ 371 Date: Aug. 25, 2017

Related U.S. Application Data
(60) Provisional application No. 62/126,439, filed on Feb. 27, 2015.

(51) Int. Cl. C12N 15/113; A61K 45/06; A61K 31/7125; A61P 3/00; A61K 9/00

(52) U.S. Cl. CPC C12N 15/113; A61K 45/06; A61K 31/7125; A61P 3/00; A61K 9/0019; C12N 2320/32; C12N 2310/315; C12N 2310/321; C12N 2310/3341; C12N 2310/341; C12N 2320/31; C12N 2310/11

(57) ABSTRACT

Provided herein are methods, compounds, and compositions for reducing expression of ApoCIII mRNA and protein in a patient with Partial Lipodystrophy. Also provided herein are methods, compounds, and compositions for treating, preventing, delaying, or ameliorating Partial Lipodystrophy in a patient. Further provided herein are methods, compounds, and compositions useful to treat, prevent, delay, or ameliorate any one or more of pancreatitis, cardiovascular disease or metabolic disorder, or a symptom thereof, associated with Partial Lipodystrophy in a patient.

### SEQUENCE LISTING

The present application is being filed along with a Sequence Listing in electronic format. The Sequence Listing is provided as a file entitled BIOL0268WOSEQ_ST25.txt, created on Feb. 24, 2016 which is 12 Kb in size. The information in the electronic format of the sequence listing is incorporated herein by reference in its entirety.

### FIELD OF THE INVENTION

Provided herein are methods, compounds, and compositions for reducing expression of Apolipoprotein C-III (ApoCIII) mRNA and protein, reducing triglyceride levels and increasing high density lipoprotein (HDL) levels or HDL activity in Partial Lipodystrophy (PL) patients. Also, provided herein are compounds and compositions for use in treating Partial Lipodystrophy or associated disorders thereof.

### BACKGROUND

Lipoproteins are globular, micelle-like particles that consist of a non-polar core of acylglycerols and cholesteryl esters surrounded by an amphiphilic coating of protein, phospholipid and cholesterol. Lipoproteins have been classified into five broad categories on the basis of their functional and physical properties: chylomicrons, very low density lipoproteins (VLDL), intermediate density lipoproteins (IDL), low density lipoproteins (LDL), and high density lipoproteins (HDL). Chylomicrons transport dietary lipids from intestine to tissues. VLDLs, IDLs and LDLs all transport triacylglycerols and cholesterol from the liver to tissues. HDLs transport endogenous cholesterol from tissues to the liver

Apolipoprotein C-III (also called APOC3, APOC-III, ApoCIII, and APO C-III) is a constituent of HDL and of triglyceride (TG)-rich lipoproteins. Elevated ApoCIII is associated with elevated TG levels in diseases such as cardiovascular disease, metabolic syndrome, obesity, diabetes (Chan et al., Int J Clin Pract, 2008, 62:799-809; Onat et at., Atherosclerosis, 2003, 168:81-89; Mendivil et al., Circulation, 2011, 124:2065-2072; Mauger et al., J. Lipid Res, 2006. 47: 1212-1218; Chan et al., Clin Chem, 2002, 278-283; Ooi et al., Clin. Sci, 2008, 114: 611-624; Davidsson et al., J. Lipid Res, 2005. 46: 1999-2006; Sacks et al., Circulation, 2000. 102: 1886-1892; Lee et al., Arterioscler Thromb Vasc Biol, 2003, 23: 853-858) and Lipodystrophy (Kassai et al., ENDO 2015 meeting abstract e-published at https://endo.confex.com/endo/2015endo/webprogram/Paper22544.html).

Lipodystrophy syndromes are a group of rare metabolic diseases characterized by selective loss of adipose tissue that leads to ectopic fat deposition in liver and muscle and the development of insulin resistance, diabetes, dyslipidemia and fatty liver disease. These syndromes are classified according to the underlying etiology (inherited or acquired) and according to the distribution of fat loss into Generalized or Partial Lipodystrophies (Garg et al., J Clin Endocrinol Metab, 2011, 96: 3313-3325; Chan et al., Endocr Pract, 2010, 16: 310-323; Simha et al., Curr Opin Lipidol, 2006, 17(2): 162-169; Garg, N Engl J Med, 2004, 350: 1220-1234).

Current treatment for Lipodystrophies includes lifestyle modification reducing caloric intake and increasing energy expenditure via exercise. Conventional therapies used to treat severe insulin resistance (metformin, thiazolidinediones, GLP-1s, insulin), and/or high TGs (fibrates, fish oils) are not very efficacious in these patients (Chan et al., Endocr Pract, 2010, 16: 310-323).

In patients with HIV-associated Lipodystrophy, Egrifta® (tesamorelin) is commercially available to reduce excess abdominal fat (Egrifta® Package Insert, 2013).

In patients with Generalized Lipodystrophy, metabolic complications are related to leptin deficiency. A leptin replacement therapy, Myalept® (metrelephin), is commercially available for patients with Generalized Lipodystrophy but due to Myalept® associated risks in developing anti-drug neutralizing antibodies to endogenous leptin or metrelephin and lymphoma, it is available only through a risk evaluation and mitigation strategy (REMS) program, which requires prescriber and pharmacy certification and special documentation (Myalept, FDA Briefing Document, 2013; Chang et al., Endocr Pract, 2011, 17(6): 922-932).

No specific pharmacologic treatment currently exists for non-iatrogenic forms of Partial Lipodystrophy.

Accordingly, there is a need to provide patients with Lipodystrophy novel treatment options. Antisense technology is emerging as an effective means for reducing the expression of certain gene products and may prove to be uniquely useful in a number of therapeutic, diagnostic, and research applications for the modulation of ApoCIII. We have previously disclosed compositions and methods for inhibiting ApoCIII by antisense compounds in US 20040208856 (U.S. Pat. No. 7,598,227), US 20060264395 (U.S. Pat. No. 7,750,141), WO 2004/093783, WO 2012/149495, WO 2014/127268, WO 2014/205449 and WO 2014/179626, all incorporated-by-reference herein. An antisense oligonucleotide targeting ApoCIII has been tested in Phase I and II clinical trials and is currently in Phase III trials to test it's effectiveness in Familial Chylomicronemia Syndrome (FCS) and hypertriglyceridemia patients.

### SUMMARY OF THE INVENTION

Certain embodiments provide a method of treating, preventing, delaying or ameliorating Lipodystrophy comprising administering a therapeutically effective amount of a compound comprising an ApoCIII specific inhibitor to the animal. Certain embodiments provide an ApoCIII specific inhibitor for use in treating, preventing, delaying or ameliorating Lipodystrophy. In certain embodiments, the Lipodystrophy is Generalized Lipodystrophy or Partial Lipodystrophy.

Certain embodiments provide a method of treating, preventing, delaying or ameliorating cardiovascular and/or metabolic disease or disorder, or symptom thereof, in an animal with Lipodystrophy comprising administering a therapeutically effective amount of a compound comprising an ApoCIII specific inhibitor to the animal. In certain embodiments, the compound prevents, delays or ameliorates the cardiovascular and/or metabolic disease, disorder, condition, or symptom thereof, in the animal with Lipodystrophy by decreasing TG levels, increasing HDL levels in the animal and/or improving the ratio of TG to HDL. In certain embodiments, Certain embodiments provide a method of treating, preventing, delaying or ameliorating hepatic steatosis, NALFD or NASH, or symptom thereof, in an animal with Lipodystrophy comprising administering a therapeutically effective amount of a compound comprising an ApoCIII specific inhibitor to the animal. In certain embodiments, the compound prevents, delays or ameliorates hepatic steatosis, NALFD or NASH, or symptom thereof, in the animal with Lipodystrophy by decreasing TG levels, increasing HDL levels in the animal and/or improving the ratio of TG to HDL. In certain embodiments, hepatic steatosis, NALFD or NASH, or a symptom or risk thereof, is improved. In certain embodiments, administering the therapeutically effective amount of the compound comprising the ApoCIII specific inhibitor to the animal with Lipodystrophy associated hepatic steatosis, NALFD or NASH prevents or delays progression to cirrhosis of the liver or hepatocellular carcinoma.

Certain embodiments provide a method of treating, preventing, delaying or ameliorating pancreatitis or symptom thereof, in an animal with Lipodystrophy comprising administering a therapeutically effective amount of a compound comprising an ApoCIII specific inhibitor to the animal. In certain embodiments, the compound prevents, delays or ameliorates pancreatitis, or symptom thereof, in the animal with Lipodystrophy by decreasing TG levels, increasing HDL levels in the animal and/or improving the ratio of TG to HDL. In certain embodiments, pancreatitis, or a symptom or risk thereof, is improved.

Certain embodiments provide a method of reducing TG levels in an animal with Lipodystrophy comprising administering a therapeutically effective amount of a compound comprising an ApoCIII specific inhibitor to the animal. In certain embodiments, hypertriglyceridemia, or a symptom or risk thereof, is improved.

Certain embodiments provide a method of increasing HDL levels and/or improving the ratio of TG to HDL in an animal with Lipodystrophy comprising administering a therapeutically effective amount of a compound comprising an ApoCIII specific inhibitor to the animal.

Certain embodiments provide a method of reducing fasting TG, reducing HbA1c, reducing plasma glucose, reducing liver volume, reducing an increase in liver volume and reducing hepatic steatosis in an animal with Lipodystrophy comprising administering a therapeutically effective amount of a compound comprising an ApoCIII specific inhibitor to the animal. In certain embodiments HbA1c is reduced to less than 9%, less than 8%, less than 7.5% or less than 7%. In certain embodiments, HbA1c is reduced by at least 0.2%, at least 0.5%, at least 0.7%, at least 1%, at least 1.2% or at least 1.5%.

In certain embodiments, the ApoCIII specific inhibitor is a nucleic acid, peptide, antibody, small molecule or other agent capable of inhibiting the expression of ApoCIII. In certain embodiments, the nucleic acid is an antisense compound targeting ApoCIII. In certain embodiments, the antisense compound is an antisense oligonucleotide. In certain embodiments, the antisense oligonucleotide is a modified oligonucleotide. In certain embodiments, the modified oligonucleotide has a nucleobase sequence comprising at least 8 contiguous nucleobases of ISIS 304801, AGCTTCTTGTCCAGCTTTAT (SEQ ID NO: 3). In certain embodiments, the modified oligonucleotide consists of the nucleobase sequence of SEQ ID NO: 3. In certain embodiments, the modified oligonucleotide is at least 70%, at least 75%, at least 80%, at least 85%, at least 90%, at least 95%, at least 98% or at least 100% complementary to SEQ ID NO: 1, SEQ ID NO: 2 or SEQ ID NO: 4.

In certain embodiments, the Lipodystrophy is Generalized Lipodystrophy. In certain embodiments, the Lipodystrophy is Partial Lipodystrophy.

### DETAILED DESCRIPTION OF THE INVENTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

Unless otherwise indicated, the following terms have the following meanings:

“2′-O-methoxyethyl” (also known as 2′-MOE, 2′-O(CH₂)₂—OCH₃ and 2′-O-(2-methoxyethyl)) refers to an O-methoxy-ethyl modification of the 2′ position of a furosyl ring. A 2′-O-methoxyethyl modified sugar is a modified sugar.

“2′-O-methoxyethyl nucleotide” means a nucleotide comprising a 2′-O-methoxyethyl modified sugar moiety.

“3′ target site” refers to the nucleotide of a target nucleic acid which is complementary to the 3′-most nucleotide of a particular antisense compound.

“5′ target site” refers to the nucleotide of a target nucleic acid which is complementary to the 5′-most nucleotide of a particular antisense compound.

“5-methylcytosine” means a cytosine modified with a methyl group attached to the 5′ position. A 5-methylcytosine is a modified nucleobase.

“About” means within ±10% of a value. For example, if it is stated, “a marker may be increased by about 50%”, it is implied that the marker may be increased between 45%-55%.

“Active pharmaceutical agent” means the substance or substances in a pharmaceutical composition that provide a therapeutic benefit when administered to an individual. For example, in certain embodiments an antisense oligonucleotide targeted to ApoCIII is an active pharmaceutical agent.

“Active target region” or “target region” means a region to which one or more active antisense compounds is targeted. “Active antisense compounds” means antisense compounds that reduce target nucleic acid levels or protein levels.

“Administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

“Administering” means providing a pharmaceutical agent to an individual, and includes, but is not limited to administering by a medical professional and self-administering.

“Agent” means an active substance that can provide a therapeutic benefit when administered to an animal. “First Agent” means a therapeutic compound of the invention. For example, a first agent can be an antisense oligonucleotide targeting ApoCIII. “Second agent” means a second therapeutic compound of the invention (e.g. a second antisense oligonucleotide targeting ApoCIII) and/or a non-ApoCIII therapeutic compound.

“Amelioration” refers to a lessening of at least one indicator, sign, or symptom of an associated disease, disorder, or condition. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAi and occupancy-based compounds.

“Antisense inhibition” means the reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid. As used herein, the term “antisense oligonucleotide” encompasses pharmaceutically acceptable derivatives of the compounds described herein.

“ApoCIII”, “Apolipoprotein C-III” or “ApoC3” means any nucleic acid or protein sequence encoding ApoCIII. For example, in certain embodiments, an ApoCIII includes a DNA sequence encoding ApoCIII, a RNA sequence transcribed from DNA encoding ApoCIII (including genomic DNA comprising introns and exons), a mRNA sequence encoding ApoCIII, or a peptide sequence encoding ApoCIII.

“ApoCIII specific inhibitor” refers to any agent capable of specifically inhibiting the expression of ApoCIII mRNA and/or the expression or activity of ApoCIII protein at the molecular level. For example, ApoCIII specific inhibitors include nucleic acids (including antisense compounds), peptides, antibodies, small molecules, and other agents capable of inhibiting the expression of ApoCIII mRNA and/or ApoCIII protein. In certain embodiments, the nucleic acid is an antisense compound. In certain embodiments, the antisense compound is an oligonucleotide targeting ApoCIII. In certain embodiments, the oligonucleotide targeting ApoCIII is a modified oligonucleotide targeting ApoCIII. In certain embodiments, the oligonucleotide targeting ApoCIII has a sequence as shown in SEQ ID NO: 3 or another sequence, for example, such as those disclosed in U.S. Pat. No. 7,598,227, U.S. Pat. No. 7,750,141, PCT Publication WO 2004/093783 or WO 2012/149495, all incorporated-by-reference herein. In certain embodiments, by specifically modulating ApoCIII mRNA level and/or ApoCIII protein expression, ApoCIII specific inhibitors may affect components of the lipogenic pathway.

Similarly, in certain embodiments, ApoCIII specific inhibitors may affect other molecular processes in an animal.

“ApoCIII mRNA” means a mRNA encoding an ApoCIII protein.

“ApoCIII protein” means any protein sequence encoding ApoCIII.

“Atherosclerosis” means a hardening of the arteries affecting large and medium-sized arteries and is characterized by the presence of fatty deposits. The fatty deposits are called “atheromas” or “plaques,” which consist mainly of cholesterol and other fats, calcium and scar tissue, and damage the lining of arteries.

“Bicyclic sugar” means a furosyl ring modified by the bridging of two non-geminal ring atoms. A bicyclic sugar is a modified sugar.

“Bicyclic nucleic acid” or “BNA” refers to a nucleoside or nucleotide wherein the furanose portion of the nucleoside or nucleotide includes a bridge connecting two carbon atoms on the furanose ring, thereby forming a bicyclic ring system.

“Cap structure” or “terminal cap moiety” means chemical modifications, which have been incorporated at either terminus of an antisense compound.

“Cardiovascular disease” or “cardiovascular disorder” refers to a group of conditions related to the heart, blood vessels, or the circulation. Examples of cardiovascular diseases include, but are not limited to, aneurysm, angina, arrhythmia, atherosclerosis, cerebrovascular disease (stroke), coronary heart disease, hypertension, dyslipidemia, hyperlipidemia, hypertriglyceridemia and hypercholesterolemia.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Cholesterol” is a sterol molecule found in the cell membranes of all animal tissues. Cholesterol must be transported in an animal's blood plasma by lipoproteins including very low density lipoprotein (VLDL), intermediate density lipoprotein (IDL), low density lipoprotein (LDL), and high density lipoprotein (HDL). “Plasma cholesterol” refers to the sum of all lipoproteins (VDL, IDL, LDL, HDL) esterified and/or non-esterified cholesterol present in the plasma or serum.

“Cholesterol absorption inhibitor” means an agent that inhibits the absorption of exogenous cholesterol obtained from diet.

“Co-administration” means administration of two or more agents to an individual. The two or more agents can be in a single pharmaceutical composition, or can be in separate pharmaceutical compositions. Each of the two or more agents can be administered through the same or different routes of administration. Co-administration encompasses parallel or sequential administration.

“Complementarity” means the capacity for pairing between nucleobases of a first nucleic acid and a second nucleic acid. In certain embodiments, complementarity between the first and second nucleic acid can be between two DNA strands, between two RNA strands, or between a DNA and an RNA strand. In certain embodiments, some of the nucleobases on one strand are matched to a complementary hydrogen bonding base on the other strand. In certain embodiments, all of the nucleobases on one strand are matched to a complementary hydrogen bonding base on the other strand. In certain embodiments, a first nucleic acid is an antisense compound and a second nucleic acid is a target nucleic acid. In certain such embodiments, an antisense oligonucleotide is a first nucleic acid and a target nucleic acid is a second nucleic acid.

“Contiguous nucleobases” means nucleobases immediately adjacent to each other.

“Constrained ethyl” or “cEt” refers to a bicyclic nucleoside having a furanosyl sugar that comprises a methyl(methyleneoxy) (4′-CH(CH₃)—O-2′) bridge between the 4′ and the 2′ carbon atoms.

“Cross-reactive” means an oligomeric compound targeting one nucleic acid sequence can hybridize to a different nucleic acid sequence. For example, in some instances an antisense oligonucleotide targeting human ApoCIII can cross-react with a murine ApoCIII. Whether an oligomeric compound cross-reacts with a nucleic acid sequence other than its designated target depends on the degree of complementarity the compound has with the non-target nucleic acid sequence. The higher the complementarity between the oligomeric compound and the non-target nucleic acid, the more likely the oligomeric compound will cross-react with the nucleic acid.

“Cure” means a method that restores health or a prescribed treatment for an illness.

“Coronary heart disease (CHD)” means a narrowing of the small blood vessels that supply blood and oxygen to the heart, which is often a result of atherosclerosis.

“Deoxyribonucleotide” means a nucleotide having a hydrogen at the 2′ position of the sugar portion of the nucleotide. Deoxyribonucleotides may be modified with any of a variety of substituents.

“Diabetes mellitus” or “diabetes” is a syndrome characterized by disordered metabolism and abnormally high blood sugar (hyperglycemia) resulting from insufficient levels of insulin or reduced insulin sensitivity. The characteristic symptoms are excessive urine production (polyuria) due to high blood glucose levels, excessive thirst and increased fluid intake (polydipsia) attempting to compensate for increased urination, blurred vision due to high blood glucose effects on the eye's optics, unexplained weight loss, and lethargy.

“Diabetic dyslipidemia” or “type 2 diabetes with dyslipidemia” means a condition characterized by Type 2 diabetes, reduced HDL-C, elevated triglycerides, and elevated small, dense LDL particles.

“Diluent” means an ingredient in a composition that lacks pharmacological activity, but is pharmaceutically necessary or desirable. For example, the diluent in an injected composition may be a liquid, e.g. saline solution.

“Dyslipidemia” refers to a disorder of lipid and/or lipoprotein metabolism, including lipid and/or lipoprotein overproduction or deficiency. Dyslipidemias may be manifested by elevation of lipids such as chylomicron, cholesterol and triglycerides as well as lipoproteins such as low-density lipoprotein (LDL) cholesterol. An example of a dyslipidemia is chylomicronemia or hypertriglyceridemia.

“Dosage unit” means a form in which a pharmaceutical agent is provided, e.g. pill, tablet, or other dosage unit known in the art. In certain embodiments, a dosage unit is a vial containing lyophilized antisense oligonucleotide. In certain embodiments, a dosage unit is a vial containing reconstituted antisense oligonucleotide.

“Dose” means a specified quantity of a pharmaceutical agent provided in a single administration, or in a specified time period. In certain embodiments, a dose can be administered in one, two, or more boluses, tablets, or injections. For example, in certain embodiments where subcutaneous administration is desired, the desired dose requires a volume not easily accommodated by a single injection, therefore, two or more injections can be used to achieve the desired dose. In certain embodiments, the pharmaceutical agent is administered by infusion over an extended period of time or continuously. Doses can be stated as the amount of pharmaceutical agent per hour, day, week, or month. Doses can also be stated as mg/kg or g/kg.

“Effective amount” or “therapeutically effective amount” means the amount of active pharmaceutical agent sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

“Fibrates” are agonists of peroxisome proliferator-activated receptor-α (PPAR-α), acting via transcription factors regulating various steps in lipid and lipoprotein metabolism. By interacting with PPAR-α, fibrates recruit different cofactors and regulate gene expression. As a consequence, fibrates are effective in lowering fasting TG levels as well as post-prandial TG and TRL remnant particles. Fibrates also have modest LDL-C lowering and HDL-C raising effects. Reduction in the expression and levels of ApoC-III is a consistent effect of PPAR-α agonists (Hertz et al. J Biol Chem, 1995, 270(22):13470-13475). A 36% reduction in plasma ApoC-III levels was reported with fenofibrate treatment in the metabolic syndrome (Watts et al. Diabetes, 2003, 52:803-811).

“Fully complementary” or “100% complementary” means each nucleobase of a nucleobase sequence of a first nucleic acid has a complementary nucleobase in a second nucleobase sequence of a second nucleic acid. In certain embodiments, a first nucleic acid is an antisense compound and a second nucleic acid is a target nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap” or “gap segment” and the external regions may be referred to as “wings” or “wing segments.”

“Genetic screening” means to screen for genotypic variations or mutations in an animal. In some instances the mutation can lead to a phenotypic change in the animal. In certain instances the phenotypic change is, or leads to, a disease, disorder or condition in the animal. For example, mutations in LMNA, PPARγ, PLIN1, AKT2, CIDEC and other genes can lead to Lipodystrophy. Genetic screening can be done by any of the art known techniques, for example, sequencing of the LMNA, PPARγ, PLIN1, AKT2, CIDEC gene or mRNA to detect mutations. The sequence of the animal being screened is compared to the sequence of a normal animal to determine whether there is any mutation in the sequence. Alternatively, for example, identification of mutations in the LMNA, PPARγ, PLIN1, AKT2, CIDEC gene or mRNA can be performed using PCR amplification and gel or chip analysis.

“Glucose” is a monosaccharide used by cells as a source of energy and inflammatory intermediate. “Plasma glucose” refers to glucose present in the plasma.

“High density lipoprotein” or “HDL” refers to a macromolecular complex of lipids (cholesterol, triglycerides and phospholipids) and proteins (apolipoproteins (apo) and enzymes). The surface of HDL contains chiefly apolipoproteins A, C and E. The function of some of these apoproteins is to direct HDL from the peripheral tissues to the liver. Serum HDL levels can be affected by underlying genetic causes (Weissglas-Volkov and Pajukanta, J Lipid Res, 2010, 51:2032-2057). Epidemiological studies have indicated that increased levels of HDL protect against cardiovascular disease or coronary heart disease (Gordon et al., Am. J. Med. 1977. 62: 707-714). These effects of HDL are independent of triglyceride and LDL concentrations. In clinical practice, a low plasma HDL is more commonly associated with other disorders that increase plasma triglycerides, for example, central obesity, insulin resistance, type 2 diabetes mellitus and renal disease (chronic renal failure or nephrotic proteinuria) (Kashyap. Am. J. Cardiol. 1998. 82: 42U-48U).

“High density lipoprotein-Cholesterol” or “HDL-C” means cholesterol associated with high density lipoprotein particles. Concentration of HDL-C in serum (or plasma) is typically quantified in mg/dL or nmol/L. “HDL-C” and “plasma HDL-C” mean HDL-C in serum and plasma, respectively.

“HMG-CoA reductase inhibitor” means an agent that acts through the inhibition of the enzyme HMG-CoA reductase, such as atorvastatin, rosuvastatin, fluvastatin, lovastatin, pravastatin, and simvastatin.

“Hybridization” means the annealing of complementary nucleic acid molecules. In certain embodiments, complementary nucleic acid molecules include an antisense compound and a target nucleic acid.

“Hypercholesterolemia” means a condition characterized by elevated cholesterol or circulating (plasma) cholesterol, LDL-cholesterol and VLDL-cholesterol, as per the guidelines of the Expert Panel Report of the National Cholesterol Educational Program (NCEP) of Detection, Evaluation of Treatment of high cholesterol in adults (see, Arch. Int. Med. (1988) 148, 36-39).

“Hyperlipidemia” or “hyperlipemia” is a condition characterized by elevated serum lipids or circulating (plasma) lipids. This condition manifests an abnormally high concentration of fats. The lipid fractions in the circulating blood are cholesterol, low density lipoproteins, very low density lipoproteins, chylomicrons and triglycerides.

“Hypertriglyceridemia” means a condition characterized by elevated triglyceride levels. Hypertriglyceridemia is the consequence of increased production and/or reduced or delayed catabolism of triglyceride (TG)-rich lipoproteins: VLDL and, to a lesser extent, chylomicrons (CM). Its etiology includes primary (i.e. genetic causes) and secondary (other underlying causes such as diabetes, metabolic syndrome/insulin resistance, obesity, physical inactivity, cigarette smoking, excess alcohol and a diet very high in carbohydrates) factors or, most often, a combination of both (Yuan et al., CMAJ, 2007, 176:1113-1120). Hypertriglyceridemia is a common clinical trait associated with Lipodystrophy. Borderline high TG levels (150-199 mg/dL) are commonly found in the general population and are a common component of the metabolic syndrome/insulin resistance states. The same is true for high TG levels (200-499 mg/dL) except that as plasma TG levels increase, underlying genetic factors play an increasingly important etiologic role. Very high TG levels (≥500 mg/dL) are most often associated with elevated CM levels as well, and are accompanied by increasing risk for acute pancreatitis. The risk of pancreatitis is considered clinically significant if TG levels exceed 880 mg/dL (>10 mmol) and the European Atherosclerosis Society/European Society of Cardiology (EAS/ESC) 2011 guidelines state that actions to prevent acute pancreatitis are mandatory (Catapano et al. 2011, Atherosclerosis, 217S: S1-S44). According to the EAS/ESC 2011 guidelines, hypertriglyceridemia is the cause of approximately 10% of all cases of pancreatitis, and development of pancreatitis can occur at TG levels between 440-880 mg/dL. Based on evidence from clinical studies demonstrating that elevated TG levels are an independent risk factor for atherosclerotic CVD, the guidelines from both the National Cholesterol Education Program Adult Treatment Panel III (NCEP 2002, Circulation, 106: 3143-421) and the American Diabetes Association (ADA 2008, Diabetes Care, 31: S12-S54.) recommend a target TG level of less than 150 mg/dL to reduce cardiovascular risk.

“Identifying” or “diagnosing” an animal with a named disease, disorder or condition means identifying, by art known methods, a subject prone to, or having, the named disease, disorder or condition.

“Identifying” or “diagnosing” an animal with Lipodystrophy (General or Partial) means to identify a subject prone to, or having, Lipodystrophy. Identification of subjects with Lipodystrophy can done by an examination of the subject's medical history in conjunction with any art known screening technique e.g., genetic screening. For example, a patient with a documented medical history of fasting TG above 500 mg/dL is then screened for mutations in the genes associated with Lipodystrophy such as LMNA, PPARγ, PLIN1, AKT2, CIDEC and the like.

“Identifying” or “diagnosing” an animal with metabolic or cardiovascular disease means identifying a subject prone to, or having, a metabolic disease, a cardiovascular disease, or a metabolic syndrome; or, identifying a subject having any symptom of a metabolic disease, cardiovascular disease, or metabolic syndrome including, but not limited to, hypercholesterolemia, hyperglycemia, hyperlipidemia, hypertriglyceridemia, hypertension increased insulin resistance, decreased insulin sensitivity, above normal body weight, and/or above normal body fat content or any combination thereof. Such identification can be accomplished by any method, including but not limited to, standard clinical tests or assessments, such as measuring serum or circulating (plasma) cholesterol, measuring serum or circulating (plasma) blood-glucose, measuring serum or circulating (plasma) triglycerides, measuring blood-pressure, measuring body fat content, measuring body weight, and the like.

“Improved cardiovascular outcome” means a reduction in the occurrence of adverse cardiovascular events, or the risk thereof. Examples of adverse cardiovascular events include, without limitation, death, reinfarction, stroke, cardiogenic shock, pulmonary edema, cardiac arrest, and atrial dysrhythmia.

“Immediately adjacent” means there are no intervening elements between the immediately adjacent elements, for example, between regions, segments, nucleotides and/or nucleosides.

“Increasing HDL” or “raising HDL” means increasing the level of HDL in an animal after administration of at least one compound of the invention, compared to the HDL level in an animal not administered any compound.

“Individual” or “subject” or “animal” means a human or non-human animal selected for treatment or therapy.

“Induce”, “inhibit”, “potentiate”, “elevate”, “increase”, “decrease”, “reduce” or the like denote quantitative differences between two states. For example, “an amount effective to inhibit the activity or expression of ApoCIII” means that the level of activity or expression of ApoCIII in a treated sample will differ from the level of ApoCIII activity or expression in an untreated sample. Such terms are applied to, for example, levels of expression, and levels of activity.

“Inhibiting the expression or activity” refers to a reduction or blockade of the expression or activity of a RNA or protein and does not necessarily indicate a total elimination of expression or activity.

“Insulin resistance” is defined as the condition in which normal amounts of insulin are inadequate to produce a normal insulin response from fat, muscle and liver cells. Insulin resistance in fat cells results in hydrolysis of stored triglycerides, which elevates free fatty acids in the blood plasma. Insulin resistance in muscle reduces glucose uptake whereas insulin resistance in liver reduces glucose storage, with both effects serving to elevate blood glucose. High plasma levels of insulin and glucose due to insulin resistance often leads to metabolic syndrome and type 2 diabetes.

“Insulin sensitivity” is a measure of how effectively an individual processes glucose. An individual having high insulin sensitivity effectively processes glucose whereas an individual with low insulin sensitivity does not effectively process glucose.

“Internucleoside linkage” refers to the chemical bond between nucleosides.

“Intravenous administration” means administration into a vein.

“Linked nucleosides” means adjacent nucleosides which are bonded together.

“Lipid-lowering” means a reduction in one or more lipids in a subject. “Lipid-raising” means an increase in a lipid (e.g., HDL) in a subject. Lipid-lowering or lipid-raising can occur with one or more doses over time.

“Lipid-lowering therapy” or “lipid lowering agent” means a therapeutic regimen provided to a subject to reduce one or more lipids in a subject. In certain embodiments, a lipid-lowering therapy is provided to reduce one or more of CETP, ApoB, total cholesterol, LDL-C, VLDL-C, IDL-C, non-HDL-C, triglycerides, small dense LDL particles, and Lp(a) in a subject. Examples of lipid-lowering therapy include statins, fibrates, MTP inhibitors.

“Lipoprotein”, such as VLDL, LDL and HDL, refers to a group of proteins found in the serum, plasma and lymph and are important for lipid transport. The chemical composition of each lipoprotein differs in that the HDL has a higher proportion of protein versus lipid, whereas the VLDL has a lower proportion of protein versus lipid.

“Low density lipoprotein-cholesterol (LDL-C)” means cholesterol carried in low density lipoprotein particles. Concentration of LDL-C in serum (or plasma) is typically quantified in mg/dL or nmol/L. “Serum LDL-C” and “plasma LDL-C” mean LDL-C in the serum and plasma, respectively.

“Major risk factors” refers to factors that contribute to a high risk for a particular disease or condition. In certain embodiments, major risk factors for cardiovascular disease include, without limitation, cigarette smoking, hypertension, low HDL-C, family history of cardiovascular disease, age, and other factors disclosed herein.

“Metabolic disorder” or “metabolic disease” refers to a condition characterized by an alteration or disturbance in metabolic function. “Metabolic” and “metabolism” are terms well known in the art and generally include the whole range of biochemical processes that occur within a living organism. Metabolic disorders include, but are not limited to, hyperglycemia, prediabetes, diabetes (type 1 and type 2), obesity, insulin resistance, metabolic syndrome and dyslipidemia due to type 2 diabetes.

“Metabolic syndrome” means a condition characterized by a clustering of lipid and non-lipid cardiovascular risk factors of metabolic origin. In certain embodiments, metabolic syndrome is identified by the presence of any 3 of the following factors: waist circumference of greater than 102 cm in men or greater than 88 cm in women; serum triglyceride of at least 150 mg/dL; HDL-C less than 40 mg/dL in men or less than 50 mg/dL in women; blood pressure of at least 130/85 mmHg; and fasting glucose of at least 110 mg/dL. These determinants can be readily measured in clinical practice (JAMA, 2001, 285: 2486-2497).

“Mismatch” or “non-complementary nucleobase” refers to the case when a nucleobase of a first nucleic acid is not capable of pairing with the corresponding nucleobase of a second or target nucleic acid.

“Mixed dyslipidemia” means a condition characterized by elevated cholesterol and elevated triglycerides.

“Modified internucleoside linkage” refers to a substitution or any change from a naturally occurring internucleoside bond. For example, a phosphorothioate linkage is a modified internucleoside linkage.

“Modified nucleobase” refers to any nucleobase other than adenine, cytosine, guanine, thymidine, or uracil. For example, 5-methylcytosine is a modified nucleobase. An “unmodified nucleobase” means the purine bases adenine (A) and guanine (G), and the pyrimidine bases thymine (T), cytosine (C), and uracil (U).

“Modified nucleoside” means a nucleoside having at least one modified sugar moiety, and/or modified nucleobase.

“Modified nucleotide” means a nucleotide having at least one modified sugar moiety, modified internucleoside linkage and/or modified nucleobase.

“Modified oligonucleotide” means an oligonucleotide comprising at least one modified nucleotide.

“Modified sugar” refers to a substitution or change from a natural sugar. For example, a 2′-O-methoxyethyl modified sugar is a modified sugar.

“Motif” means the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“Nicotinic acid” or “niacin” has been reported to decrease fatty acid influx to the liver and the secretion of VLDL by the liver. This effect appears to be mediated in part by the effects on hormone-sensitive lipase in the adipose tissue. Nicotinic acid has key action sites in both liver and adipose tissue. In the liver, nicotinic acid is reported to inhibit diacylglycerol acyltransferase-2 (DGAT-2) that results in the decreased secretion of VLDL particles from the liver, which is also reflected in reductions of both IDL and LDL particles, in addition, nicotinic acid raises HDL-C and apo A1 primarily by stimulating apo A1 production in the liver and has also been shown to reduce VLDL-ApoCIII concentrations in patients with hyperlipidemia (Wahlberg et al. Acta Med Scand 1988; 224:319-327). The effects of nicotinic acid on lipolysis and fatty acid mobilization in adipocytes are well established.

“Nucleic acid” refers to molecules composed of monomeric nucleotides. A nucleic acid includes ribonucleic acids (RNA), deoxyribonucleic acids (DNA), single-stranded nucleic acids (ssDNA), double-stranded nucleic acids (dsDNA), small interfering ribonucleic acids (siRNA), and microRNAs (miRNA). A nucleic acid may also comprise a combination of these elements in a single molecule.

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the oligonucleotide and the target nucleic acid are considered to be complementary at that nucleobase pair.

“Nucleobase sequence” means the order of contiguous nucleobases independent of any sugar, linkage, or nucleobase modification.

“Nucleoside” means a nucleobase linked to a sugar.

“Nucleoside mimetic” includes those structures used to replace the sugar or the sugar and the base, and not necessarily the linkage at one or more positions of an oligomeric compound; for example nucleoside mimetics having morpholino, cyclohexenyl, cyclohexyl, tetrahydropyranyl, bicyclo or tricyclo sugar mimetics such as non-furanose sugar units.

“Nucleotide” means a nucleoside having a phosphate group covalently linked to the sugar portion of the nucleoside.

“Nucleotide mimetic” includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

“Oligomeric compound” or “oligomer” means a polymer of linked monomeric subunits which is capable of hybridizing to a region of a nucleic acid molecule. In certain embodiments, oligomeric compounds are oligonucleosides. In certain embodiments, oligomeric compounds are oligonucleotides. In certain embodiments, oligomeric compounds are antisense compounds. In certain embodiments, oligomeric compounds are antisense oligonucleotides. In certain embodiments, oligomeric compounds are chimeric oligonucleotides.

“Oligonucleotide” means a polymer of linked nucleosides each of which can be modified or unmodified, independent from one another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration. Administration can be continuous, chronic, short or intermittent.

“Peptide” means a molecule formed by linking at least two amino acids by amide bonds. Peptide refers to polypeptides and proteins.

“Pharmaceutical agent” means a substance that provides a therapeutic benefit when administered to an individual. For example, in certain embodiments, an antisense oligonucleotide targeted to ApoCIII is pharmaceutical agent.

“Pharmaceutical composition” or “composition” means a mixture of substances suitable for administering to an individual. For example, a pharmaceutical composition may comprise one or more active agents and a pharmaceutical carrier, such as a sterile aqueous solution.

“Pharmaceutically acceptable carrier” means a medium or diluent that does not interfere with the structure of the compound. Certain of such carriers enable pharmaceutical compositions to be formulated as, for example, tablets, pills, dragees, capsules, liquids, gels, syrups, slurries, suspension and lozenges for the oral ingestion by a subject. Certain of such carriers enable pharmaceutical compositions to be formulated for injection, infusion or topical administration. For example, a pharmaceutically acceptable carrier can be a sterile aqueous solution.

“Pharmaceutically acceptable derivative” or “salts” encompasses derivatives of the compounds described herein such as solvates, hydrates, esters, prodrugs, polymorphs, isomers, isotopically labelled variants, pharmaceutically acceptable salts and other derivatives known in the art.

“Pharmaceutically acceptable salts” means physiologically and pharmaceutically acceptable salts of antisense compounds, i.e., salts that retain the desired biological activity of the parent compound and do not impart undesired toxicological effects thereto. The term “pharmaceutically acceptable salt” or “salt” includes a salt prepared from pharmaceutically acceptable non-toxic acids or bases, including inorganic or organic acids and bases. Pharmaceutically acceptable salts of the compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002). Sodium salts of antisense oligonucleotides are useful and are well accepted for therapeutic administration to humans. Accordingly, in one embodiment the compounds described herein are in the form of a sodium salt.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form (i.e., a drug) within the body or cells thereof by the action of endogenous enzymes or other chemicals or conditions.

“Raise” means to increase in amount. For example, to raise plasma HDL levels means to increase the amount of HDL in the plasma.

“Ratio of TG to HDL” means the TG levels relative to HDL levels. The occurrence of high TG and/or low HDL has been linked to cardiovascular disease incidence, outcomes and mortality. “Improving the ratio of TG to HDL” means to decrease TG and/or raise HDL levels.

“Reduce” means to bring down to a smaller extent, size, amount, or number. For example, to reduce plasma triglyceride levels means to bring down the amount of triglyceride in the plasma.

“Region” or “target region” is defined as a portion of the target nucleic acid having at least one identifiable structure, function, or characteristic. For example, a target region may encompass a 3′ UTR, a 5′ UTR, an exon, an intron, an exon/intron junction, a coding region, a translation initiation region, translation termination region, or other defined nucleic acid region. The structurally defined regions for ApoCIII can be obtained by accession number from sequence databases such as NCBI and such information is incorporated herein by reference. In certain embodiments, a target region may encompass the sequence from a 5′ target site of one target segment within the target region to a 3′ target site of another target segment within the target region.

“Ribonucleotide” means a nucleotide having a hydroxy at the 2′ position of the sugar portion of the nucleotide. Ribonucleotides can be modified with any of a variety of substituents.

“Second agent” or “second therapeutic agent” means an agent that can be used in combination with a “first agent”. A second therapeutic agent can include, but is not limited to, an siRNA or antisense oligonucleotide including antisense oligonucleotides targeting ApoCIII. A second agent can also include leptin replacement therapy, anti-ApoCIII antibodies, ApoCIII peptide inhibitors, DGAT1 inhibitors, cholesterol lowering agents, lipid lowering agents, glucose lowering agents and anti-inflammatory agents.

“Segments” are defined as smaller, sub-portions of regions within a nucleic acid. For example, a “target segment” means the sequence of nucleotides of a target nucleic acid to which one or more antisense compounds is targeted. “5′ target site” refers to the 5′-most nucleotide of a target segment. “3′ target site” refers to the 3′-most nucleotide of a target segment.

“Shortened” or “truncated” versions of antisense oligonucleotides or target nucleic acids taught herein have one, two or more nucleosides deleted.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity to a target nucleic acid to induce a desired effect, while exhibiting minimal or no effects on non-target nucleic acids under conditions in which specific binding is desired, i.e. under physiological conditions in the case of in vivo assays and therapeutic treatments.

“Statin” means an agent that inhibits the activity of HMG-CoA reductase. Statins reduce synthesis of cholesterol in the liver by competitively inhibiting HMG-CoA reductase activity.

The reduction in intracellular cholesterol concentration induces LDL receptor expression on the hepatocyte cell surface, which results in increased extraction of LDL-C from the blood and a decreased concentration of circulating LDL-C and other apo-B containing lipoproteins including TG-rich particles. Independent of their effects on LDL-C and LDL receptor, statins lower the plasma concentration and cellular mRNA levels of ApoC-III (Ooi et al. Clinical Sci, 2008, 114:611-624). As statins have significant effects on mortality as well as most cardiovascular disease outcome parameters, these drugs are the first choice to reduce both total cardiovascular disease risk and moderately elevated TG levels. More potent statins (atorvastatin, rosuvastatin, and pitavastatin) demonstrate a robust lowering of TG levels, especially at high doses and in patients with elevated TG.

“Subcutaneous administration” means administration just below the skin.

“Subject” means a human or non-human animal selected for treatment or therapy.

“Symptom of cardiovascular disease or disorder” means a phenomenon that arises from and accompanies the cardiovascular disease or disorder and serves as an indication of it. For example, angina; chest pain; shortness of breath; palpitations; weakness; dizziness; nausea; sweating; tachycardia; bradycardia; arrhythmia; atrial fibrillation; swelling in the lower extremities; cyanosis; fatigue; fainting; numbness of the face; numbness of the limbs; claudication or cramping of muscles; bloating of the abdomen; or fever are symptoms of cardiovascular disease or disorder.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Therapeutic lifestyle change” means dietary and lifestyle changes intended to lower fat/adipose tissue mass and/or cholesterol. Such change can reduce the risk of developing heart disease, and may includes recommendations for dietary intake of total daily calories, total fat, saturated fat, polyunsaturated fat, monounsaturated fat, carbohydrate, protein, cholesterol, insoluble fiber, as well as recommendations for physical activity.

“Treat” refers to administering a compound of the invention to effect an alteration or improvement of a disease, disorder, or condition.

“Triglyceride” or “TG” means a lipid or neutral fat consisting of glycerol combined with three fatty acid molecules.

“Type 2 diabetes,” (also known as “type 2 diabetes mellitus”, “diabetes mellitus, type 2”, “non-insulin-dependent diabetes (NIDDM)”, “obesity related diabetes”, or “adult-onset diabetes”) is a metabolic disorder that is primarily characterized by insulin resistance, relative insulin deficiency, and hyperglycemia.

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

“Wing segment” means one or a plurality of nucleosides modified to impart to an oligonucleotide properties such as enhanced inhibitory activity, increased binding affinity for a target nucleic acid, or resistance to degradation by in vivo nucleases.

### Certain Embodiments

Certain embodiments provide a method of reducing ApoCIII levels in an animal with Lipodystrophy comprising administering a therapeutically effective amount of a compound comprising an ApoCIII specific inhibitor to the animal. In certain embodiments, ApoCIII levels are reduced in the liver, adipose tissue, heart, skeletal muscle or small intestine.

In certain embodiments, the Lipodystrophy is Generalized Lipodystrophy or Partial Lipodystrophy.

Certain embodiments provide a method of treating, preventing, delaying or ameliorating Lipodystrophy in an animal comprising administering a therapeutically effective amount of a compound comprising an ApoCIII specific inhibitor to the animal. In certain embodiments, Lipodystrophy, or a symptom or risk thereof, is improved.

Certain embodiments provide a method of treating, preventing, delaying or ameliorating cardiovascular and/or metabolic disease or disorder, or symptom thereof, in an animal with Lipodystrophy comprising administering a therapeutically effective amount of a compound comprising an ApoCIII specific inhibitor to the animal. In certain embodiments, the compound prevents, delays or ameliorates the cardiovascular and/or metabolic disease, disorder, condition, or symptom thereof, in the animal with Lipodystrophy by decreasing TG levels, increasing HDL levels in the animal and/or improving the ratio of TG to HDL. In certain embodiments, cardiovascular and/or metabolic disease or disorder, or a symptom or risk thereof, is improved.

In certain embodiments, the cardiovascular disease is aneurysm, angina, arrhythmia, atherosclerosis, cerebrovascular disease, coronary heart disease, hypertension, dyslipidemia, hyperlipidemia, hypertriglyceridemia or hypercholesterolemia. In certain embodiments, the dyslipidemia is hypertriglyceridemia or chylomicronemia. In certain embodiments, the metabolic disease is diabetes, obesity or metabolic syndrome.

In certain embodiments, symptoms of a cardiovascular disease include, but are not limited to, angina; chest pain; shortness of breath; palpitations; weakness; dizziness; nausea; sweating; tachycardia; bradycardia; arrhythmia; atrial fibrillation; swelling in the lower extremities; cyanosis; fatigue; fainting; numbness of the face; numbness of the limbs; claudication or cramping of muscles; bloating of the abdomen; or fever.

Certain embodiments provide a method of treating, preventing, delaying or ameliorating hepatic steatosis, NALFD or NASH, or symptom thereof, in an animal with Lipodystrophy comprising administering a therapeutically effective amount of a compound comprising an ApoCIII specific inhibitor to the animal. In certain embodiments, the compound prevents, delays or ameliorates hepatic steatosis, NALFD or NASH, or symptom thereof, in the animal with Lipodystrophy by decreasing TG levels, increasing HDL levels in the animal and/or improving the ratio of TG to HDL. In certain embodiments, hepatic steatosis, NALFD or NASH, or a symptom or risk thereof, is improved. In certain embodiments, administering the therapeutically effective amount of the compound comprising the ApoCIII specific inhibitor to the animal with Lipodystrophy associated hepatic steatosis, NALFD or NASH prevents or delays progression to cirrhosis of the liver or hepatocellular carcinoma.

Certain embodiments provide a method of treating, preventing, delaying or ameliorating pancreatitis or symptom thereof, in an animal with Lipodystrophy comprising administering a therapeutically effective amount of a compound comprising an ApoCIII specific inhibitor to the animal. In certain embodiments, the compound prevents, delays or ameliorates pancreatitis, or symptom thereof, in the animal with Lipodystrophy by decreasing TG levels, increasing HDL levels in the animal and/or improving the ratio of TG to HDL. In certain embodiments, pancreatitis, or a symptom or risk thereof, is improved.

Certain embodiments provide a method of reducing TG levels in an animal with Lipodystrophy comprising administering a therapeutically effective amount of a compound comprising an ApoCIII specific inhibitor to the animal. In certain embodiments, hypertriglyceridemia, or a symptom or risk thereof, is improved.

In certain embodiments, the animal has a TG level of at least ≥1200 mg/dL, ≥1100 mg/dL, ≥1000 mg/dL, ≥900 mg/dL, ≥880 mg/dL, ≥850 mg/dL, ≥800 mg/dL, ≥750 mg/dL, ≥700 mg/dL, ≥650 mg/dL, ≥600 mg/dL, ≥550 mg/dL, ≥500 mg/dL, ≥450 mg/dL, ≥440 mg/dL, ≥400 mg/dL, ≥350 mg/dL, ≥300 mg/dL, ≥250 mg/dL, ≥200 mg/dL, ≥150 mg/dL In certain embodiments, the animal has a history of TG level ≥880 mg/dL, fasting TG level ≥750 mg/dL and/or TG level ≥440 mg/dL after dieting.

In certain embodiments, the compound decreases TGs (postprandial or fasting) by at least 90%, by at least 80%, by at least 70%, by at least 60%, by at least 50%, by at least 45%, at least 40%, by at least 35%, by at least 30%, by at least 25%, by at least 20%, by at least 15%, by at least 10%, by at least 5% or by at least 1% from the baseline TG level. In certain embodiments, the TG (postprandial or fasting) level is ≤1900 mg/dL, ≤1800 mg/dL, ≤1700 mg/dL, ≤1600 mg/dL, ≤1500 mg/dL, ≤1400 mg/dL, ≤1300 mg/dL, ≤1200 mg/dL, ≤1100 mg/dL, ≤1000 mg/dL, ≤900 mg/dL, ≤800 mg/dL, ≤750 mg/dL, ≤700 mg/dL, ≤650 mg/dL, ≤600 mg/dL, ≤550 mg/dL, ≤500 mg/dL, ≤450 mg/dL, ≤400 mg/dL, ≤350 mg/dL, ≤300 mg/dL, ≤250 mg/dL, ≤200 mg/dL, ≤150 mg/dL or ≤100 mg/dL.

Certain embodiments provide a method of increasing HDL levels and/or improving the ratio of TG to HDL in an animal with Lipodystrophy comprising administering a therapeutically effective amount of a compound comprising an ApoCIII specific inhibitor to the animal. In certain embodiments, the compound increases HDL (postprandial or fasting) by at least 90%, by at least 80%, by at least 70%, by at least 60%, by at least 50%, by at least 45%, at least 40%, by at least 35%, by at least 30%, by at least 25%, by at least 20%, by at least 15%, by at least 10%, by at least 5% or by at least 1% from the baseline HDL level.

Certain embodiments provide a method of reducing fasting TG, reducing HbA1c, reducing plasma glucose, reducing liver volume, reducing an increase in liver volume and reducing hepatic steatosis in an animal with Lipodystrophy comprising administering a therapeutically effective amount of a compound comprising an ApoCIII specific inhibitor to the animal. In certain embodiments HbA1c is reduced to less than 9%, less than 8%, less than 7.5% or less than 7%. In certain embodiments, HbA1c is reduced by at least 0.2%, at least 0.5%, at least 0.7%, at least 1%, at least 1.2% or at least 1.5%.

Additional embodiments provide a method to improve physiological markers such as glycemic indicators, lipid parameters, adipose tissue parameters and patient reported outcomes in a patient with Lipodystrophy comprising administering a therapeutically effective amount of a compound comprising an ApoCIII specific inhibitor to the patient.

Examples of glycemic indicators to improve include, but are not limited to, glucose levels, homeostatic model assessment (HOMA), insulin resistance, fasting insulin levels, C-peptide levels and insulin usage. In certain embodiments, it is desirable to

Examples of lipids parameters to improve include, but are not limited to, HDL-C, LDL-C, total cholesterol, VLDL-C, non-HDL-C, apoB, apoA1, apoC3 (total, chylomicron, VLDL, LDL and HDL), free fatty acids and/or lipoprotein particle size and/or number will be assessed for improvement.

Examples of adipose tissue parameters to improve include, but are not limited to, skinfold thickness, percentage body fat (DEXA scan), adiponectin, leptin, body weight and waist circumference.

Examples of patient reported outcomes parameters to improve include, but are not limited to, Quality of Life (EQ-5D, SF36 surveys) and hunger scale.

Certain embodiments provide a method for treating patients with Lipodystrophy suffering from severe or multiple pancreatitis attacks comprising comprising administering a therapeutically effective amount of a compound comprising an ApoCIII specific inhibitor to the patient. In certain embodiments, the patient suffers from pancreatitis despite dietary fat restrictions.

Certain embodiments provide a method for identifying a subject suffering from Lipodystrophy comprising genetically screening the subject. Certain embodiments provide a method for identifying a subject at risk for Lipodystrophy comprising genetically screening the subject. In certain embodiments the genetic screening is performed by sequence analysis of the gene or RNA transcript encoding LMNA, PPARγ, PLIN1, AKT2, CIDEC or any other gene or RNA associated with Lipodystrophy.

Certain embodiments provide a method for identifying a subject suffering from Lipodystrophy comprising screening the subject by clinical assessment and/or genetic screening.

In certain embodiments, the ApoCIII nucleic acid is any of the sequences set forth in GENBANK Accession No. NM 000040.1 (incorporated herein as SEQ ID NO: 1), GENBANK Accession No. NT 033899.8 truncated from nucleotides 20262640 to 20266603 (incorporated herein as SEQ ID NO: 2), and GenBank Accession No. NT_035088.1 truncated from nucleotides 6238608 to U.S. Pat. No. 6,242,565 (incorporated herein as SEQ ID NO: 4).

In certain embodiments, the ApoCIII specific inhibitor comprises a nucleic acid, peptide, antibody, small molecule or other agent capable of inhibiting the expression of ApoCIII. In certain embodiments, the nucleic acid comprises an antisense compound targeting ApoCIII. In certain embodiments, the antisense compound comprises an antisense oligonucleotide targeting ApoCIII. In certain embodiments, the antisense oligonucleotide comprises a modified oligonucleotide targeting ApoCIII. In certain embodiments, the modified oligonucleotide has a sequence complementary to SEQ ID NO: 1, SEQ ID NO: 2 or SEQ ID NO: 4. In certain embodiments, the modified oligonucleotide is at least 70%, at least 75%, at least 80%, at least 85%, at least 90%, at least 95%, at least 98% or 100% complementary to SEQ ID NO: 1, SEQ ID NO: 2 or SEQ ID NO: 4.

In certain embodiments, the modified oligonucleotide has a nucleobase sequence comprising at least 8 contiguous nucleobases of an antisense oligonucleotide complementary to an ApoCIII. In certain embodiments, the modified oligonucleotide has a nucleobase sequence comprising at least 8 contiguous nucleobases of ISIS 304801 (SEQ ID NO: 3). In certain embodiments, the modified oligonucleotide has a nucleobase sequence of ISIS 304801 (SEQ ID NO: 3). In certain embodiments, the modified oligonucleotide targeting ApoCIII has a sequence other than that of SEQ ID NO: 3. In certain embodiments, the modified oligonucleotide has a nucleobase sequence comprising at least 8 contiguous nucleobases of a sequence selected from any sequence disclosed in U.S. Pat. No. 7,598,227, U.S. Pat. No. 7,750,141, PCT Publication WO 2004/093783 or PCT Publication WO 2012/149495, all incorporated-by-reference herein. In certain embodiments, the modified oligonucleotide has a sequence selected from any sequence disclosed in U.S. Pat. No. 7,598,227, U.S. Pat. No. 7,750,141, PCT Publication WO 2004/093783 or PCT Publication WO 2012/149495, all incorporated-by-reference herein.

In certain embodiments, the modified oligonucleotide consists of a single-stranded modified oligonucleotide.

In certain embodiments, the modified oligonucleotide consists of 12-30 linked nucleosides. In certain embodiments, the modified oligonucleotide consists of 19-22 linked nucleosides. In certain embodiments, the modified oligonucleotide consists of 20 linked nucleosides. In certain embodiments, the modified oligonucleotide consists of 20 linked nucleosides and the nucleobase sequence of ISIS 304801 (SEQ ID NO: 3).

In certain embodiments, the compound comprises at least one modified internucleoside linkage. In certain embodiments, the internucleoside linkage is a phosphorothioate internucleoside linkage. In certain embodiments, each internucleoside linkage is a phosphorothioate internucleoside linkage.

In certain embodiments, the compound comprises at least one nucleoside comprising a modified sugar. In certain embodiments, the at least one modified sugar is a bicyclic sugar. In certain embodiments, the at least one modified sugar comprises a 2′-O-methoxyethyl.

In certain embodiments, the compound comprises at least one nucleoside comprising a modified nucleobase. In certain embodiments, the modified nucleobase is a 5-methylcytosine.

In certain embodiments, the compound comprises a modified oligonucleotide comprising: (i) a gap segment consisting of linked deoxynucleosides; (ii) a 5′ wing segment consisting of linked nucleosides; (iii) a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In certain embodiments, the compound comprises a modified oligonucleotide comprising: (i) a gap segment consisting of 8-12 linked deoxynucleosides; (ii) a 5′ wing segment consisting of 1-5 linked nucleosides; (iii) a 3′ wing segment consisting of 1-5 linked nucleosides, wherein the gap segment is positioned immediately adjacent to and between the 5′ wing segment and the 3′ wing segment, wherein each nucleoside of each wing segment comprises a 2′-O-methoxyethyl sugar, wherein each cytosine is a 5′-methylcytosine, and wherein at least one internucleoside linkage is a phosphorothioate linkage. In certain embodiments each internucleoside linkage is a phosphorothioate linkage

In certain embodiments, the compound comprises a modified oligonucleotide comprising: (i) a gap segment consisting of ten linked deoxynucleosides; (ii) a 5′ wing segment consisting of five linked nucleosides; (iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar, wherein each cytosine is a 5′-methylcytosine, and wherein at least one internucleoside linkage is a phosphorothioate linkage. In certain embodiments each internucleoside linkage is a phosphorothioate linkage.

Certain embodiments provide a method of treating, preventing, delaying or ameliorating Partial Lipodystrophy, or a disease associated with Partial Lipodystophy in an animal comprising administering to the animal a therapeutically effective amount of a compound comprising a modified oligonucleotide having the sequence of SEQ ID NO: 3 wherein the modified oligonucleotide comprises: (i) a gap segment consisting of ten linked deoxynucleosides; (ii) a 5′ wing segment consisting of five linked nucleosides; (iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar, wherein each cytosine is a 5′-methylcytosine, and wherein at least one internucleoside linkage is a phosphorothioate linkage. In certain embodiments each internucleoside linkage is a phosphorothioate linkage.

Certain embodiments provide a method of treating, preventing, delaying or ameliorating Partial Lipodystrophy, or a disease associated with Partial Lipodystophy in an animal comprising administering to the animal a therapeutically effective amount of a compound comprising a modified oligonucleotide consisting of 12 to 30 linked nucleosides, wherein the modified oligonucleotide is complementary to an ApoCIII nucleic acid and wherein the modified oligonucleotide decreases TG levels, increases HDL levels and/or improves the ratio of TG to HDL. In certain embodiments, the ApoCIII nucleic acid is SEQ ID NO: 1, SEQ ID NO: 2 or SEQ ID NO: 4. In certain embodiments, the modified oligonucleotide is at least 70%, least 75%, least 80%, at least 85%, at least 90%, at least 95%, at least 98% or 100% complementary to SEQ ID NO: 1, SEQ ID NO: 2 or SEQ ID NO: 4. In certain embodiments, the modified oligonucleotide comprises at least 8 contiguous nucleobases of an antisense oligonucleotide targeting ApoCIII. In further embodiments, the modified oligonucleotide comprises at least 8 contiguous nucleobases of the nucleobase sequence of ISIS 304801 (SEQ ID NO: 3).

Certain embodiments provide a method of reducing triglyceride levels in an animal with Partial Lipodystrophy comprising administering to the animal a therapeutically effective amount of a compound comprising a modified oligonucleotide having the sequence of SEQ ID NO: 3 wherein the modified oligonucleotide comprises: (i) a gap segment consisting of ten linked deoxynucleosides; (ii) a 5′ wing segment consisting of five linked nucleosides; (iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar, wherein each cytosine is a 5′-methylcytosine, and wherein at least one internucleoside linkage is a phosphorothioate linkage. In certain embodiments each internucleoside linkage is a phosphorothioate linkage.

Certain embodiments provide a method of reducing triglyceride levels in an animal with Partial Lipodystrophy comprising administering to the animal a therapeutically effective amount of a compound comprising a modified oligonucleotide consisting of 12 to 30 linked nucleosides, wherein the modified oligonucleotide is complementary to an ApoCIII nucleic acid and wherein the modified oligonucleotide decreases TG levels, increases HDL levels and/or improves the ratio of TG to HDL. In certain embodiments, the ApoCIII nucleic acid is SEQ ID NO: 1, SEQ ID NO: 2 or SEQ ID NO: 4. In certain embodiments, the modified oligonucleotide is at least 70%, least 75%, least 80%, at least 85%, at least 90%, at least 95%, at least 98% or 100% complementary to SEQ ID NO: 1, SEQ ID NO: 2 or SEQ ID NO: 4. In certain embodiments, the modified oligonucleotide comprises at least 8 contiguous nucleobases of an antisense oligonucleotide targeting ApoCIII. In further embodiments, the modified oligonucleotide comprises at least 8 contiguous nucleobases of the nucleobase sequence of ISIS 304801 (SEQ ID NO: 3).

Certain embodiments provide a method of preventing, delaying or ameliorating a cardiovascular and/or metabolic disease, disorder, condition, or symptom thereof, in an animal with Partial Lipodystrophy comprising administering to the animal a therapeutically effective amount of a compound comprising a modified oligonucleotide having the sequence of SEQ ID NO: 3 wherein the modified oligonucleotide comprises: (i) a gap segment consisting of ten linked deoxynucleosides; (ii) a 5′ wing segment consisting of five linked nucleosides; (iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar, wherein each cytosine is a 5′-methylcytosine, and wherein at least one internucleoside linkage is a phosphorothioate linkage. In certain embodiments each internucleoside linkage is a phosphorothioate linkage.

Certain embodiments provide a method of preventing, delaying or ameliorating a cardiovascular and/or metabolic disease, disorder, condition, or symptom thereof, in an animal with Partial Lipodystrophy comprising administering to the animal a therapeutically effective amount of a compound comprising a modified oligonucleotide consisting of 12 to 30 linked nucleosides, wherein the modified oligonucleotide is complementary to an ApoCIII nucleic acid and wherein the modified oligonucleotide decreases TG levels, increases HDL levels and/or improves the ratio of TG to HDL. In certain embodiments, the ApoCIII nucleic acid is SEQ ID NO: 1, SEQ ID NO: 2 or SEQ ID NO: 4. In certain embodiments, the modified oligonucleotide is at least 70%, least 75%, least 80%, at least 85%, at least 90%, at least 95%, at least 98% or 100% complementary to SEQ ID NO: 1, SEQ ID NO: 2 or SEQ ID NO: 4. In certain embodiments, the modified oligonucleotide comprises at least 8 contiguous nucleobases of an antisense oligonucleotide targeting ApoCIII. In further embodiments, the modified oligonucleotide comprises at least 8 contiguous nucleobases of the nucleobase sequence of ISIS 304801 (SEQ ID NO: 3).

Certain embodiments provide a method of preventing, delaying or ameliorating pancreatitis or symptom thereof, in an animal with Partial Lipodystrophy comprising administering to the animal a therapeutically effective amount of a compound comprising a modified oligonucleotide having the sequence of SEQ ID NO: 3 wherein the modified oligonucleotide comprises: (i) a gap segment consisting of ten linked deoxynucleosides; (ii) a 5′ wing segment consisting of five linked nucleosides; (iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar, wherein each cytosine is a 5′-methylcytosine, and wherein at least one internucleoside linkage is a phosphorothioate linkage. In certain embodiments each internucleoside linkage is a phosphorothioate linkage.

Certain embodiments provide a method of preventing, delaying or ameliorating pancreatitis or symptom thereof, in an animal with Partial Lipodystrophy comprising administering to the animal a therapeutically effective amount of a compound comprising a modified oligonucleotide consisting of 12 to 30 linked nucleosides, wherein the modified oligonucleotide is complementary to an ApoCIII nucleic acid and wherein the modified oligonucleotide decreases TG levels, increases HDL levels and/or improves the ratio of TG to HDL. In certain embodiments, the ApoCIII nucleic acid is SEQ ID NO: 1, SEQ ID NO: 2 or SEQ ID NO: 4. In certain embodiments, the modified oligonucleotide is at least 70%, least 75%, least 80%, at least 85%, at least 90%, at least 95%, at least 98% or 100% complementary to SEQ ID NO: 1, SEQ ID NO: 2 or SEQ ID NO: 4. In certain embodiments, the modified oligonucleotide comprises at least 8 contiguous nucleobases of an antisense oligonucleotide targeting ApoCIII. In further embodiments, the modified oligonucleotide comprises at least 8 contiguous nucleobases of the nucleobase sequence of ISIS 304801 (SEQ ID NO: 3).

In certain embodiments, the animal is human.

In certain embodiments, the animal with Lipodystrophy is at risk for pancreatitis. In certain embodiments, reducing ApoCIII levels in the liver and/or small intestine prevents pancreatitis. In certain embodiments, reducing TG levels, raising HDL levels and/or improving the ratio of TG to HDL prevents pancreatitis.

In certain embodiments, reducing ApoCIII levels in the liver and/or small intestine of an animal with Lipodystrophy enhances clearance of postprandial TG. In certain embodiments, raising HDL levels and/or improving the ratio of TG to HDL enhance clearance of postprandial TG in an animal with Lipodystrophy. In certain embodiments, reducing ApoCIII levels in the liver and/or small intestine lowers postprandial triglyceride in an animal with Lipodystrophy. In certain embodiments, raising HDL levels and/or improving the ratio of TG to HDL lowers postprandial TG.

In certain embodiments, the compound is parenterally administered. In further embodiments, the parenteral administration is subcutaneous.

In certain embodiments, the compound is co-administered with a second agent or therapy. In certain embodiments, the second agent is growth hormone-releasing factor (GRF), leptin replacement agent, ApoCIII lowering agent, Apo C-II lowering agent, DGAT1 lowering agent, LPL raising agent, cholesterol lowering agent, non-HDL lipid lowering agent, LDL lowering agent, TG lowering agent, cholesterol lowering agent, HDL raising agent, fish oil, niacin (nicotinic acid), fibrate, statin, DCCR (salt of diazoxide), glucose-lowering agent or anti-diabetic agents. In certain embodiments, the second therapy is dietary fat restriction.

An example of a leptin replacement agent is Myalept®.

An example of a growth hormone-releasing factor (GRF) is Egrifta®.

In certain embodiments, the ApoCIII lowering agents include an ApoCIII antisense oligonucleotide different from the first agent, fibrate or an Apo B antisense oligonucleotide.

In certain embodiments, the DGAT1 lowering agent is LCQ908.

In certain embodiments, the LPL raising agents include gene therapy agents that raise the level of LPL (e.g., Glybera^{R}, normal copies of ApoC-II, GPIHBP1, APOA5, LMF1 or other genes that, when mutated, can lead to dysfunctional LPL).

In certain embodiments, the glucose-lowering and/or anti-diabetic agents include, but are not limited to, PPAR agonist, a dipeptidyl peptidase (IV) inhibitor, a GLP-1 analog, insulin or an insulin analog, an insulin secretagogue, a SGLT2 inhibitor, a human amylin analog, a biguanide, an alpha-glucosidase inhibitor, metformin, sulfonylurea, rosiglitazone, meglitinide, thiazolidinedione, alpha-glucosidase inhibitor and the like. The sulfonylurea can be acetohexamide, chlorpropamide, tolbutamide, tolazamide, glimepiride, a glipizide, a glyburide, or a gliclazide. The meglitinide can be nateglinide or repaglinide. The thiazolidinedione can be pioglitazone or rosiglitazone. The alpha-glucosidase can be acarbose or miglitol.

In certain embodiments, the cholesterol or lipid lowering agents include, but are not limited to, statins, bile acids sequestrants, nicotinic acid and fibrates. The statins can be atorvastatin, fluvastatin, lovastatin, pravastatin, rosuvastatin and simvastatin and the like. The bile acid sequestrants can be colesevelam, cholestyramine, colestipol and the like. The fibrates can be gemfibrozil, fenofibrate, clofibrate and the like. The therapeutic lifestyle change can be dietary fat restriction.

In certain embodiments, the HDL increasing agents include cholesteryl ester transfer protein (CETP) inhibiting drugs (such as Torcetrapib), peroxisome proliferation activated receptor agonists, Apo-A1, Pioglitazone and the like.

In certain embodiments, the compound and the second agent are administered concomitantly or sequentially.

In certain embodiments, the compound is a salt form.

In further embodiments, the compound further comprises of a pharmaceutically acceptable carrier or diluent.

In certain embodiments, the compound is conjugated. In certain embodiments, the compound is GalNAc conjugated. In certain embodiments, the compound comprises a GalNAc conjugate group with the formula:

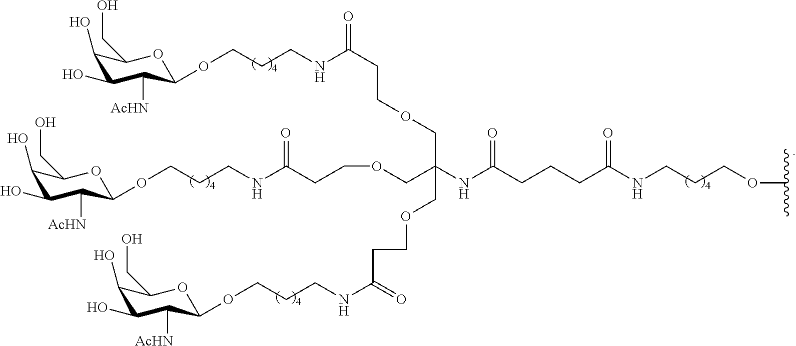

In certain embodiments, the conjugated compound has the formula

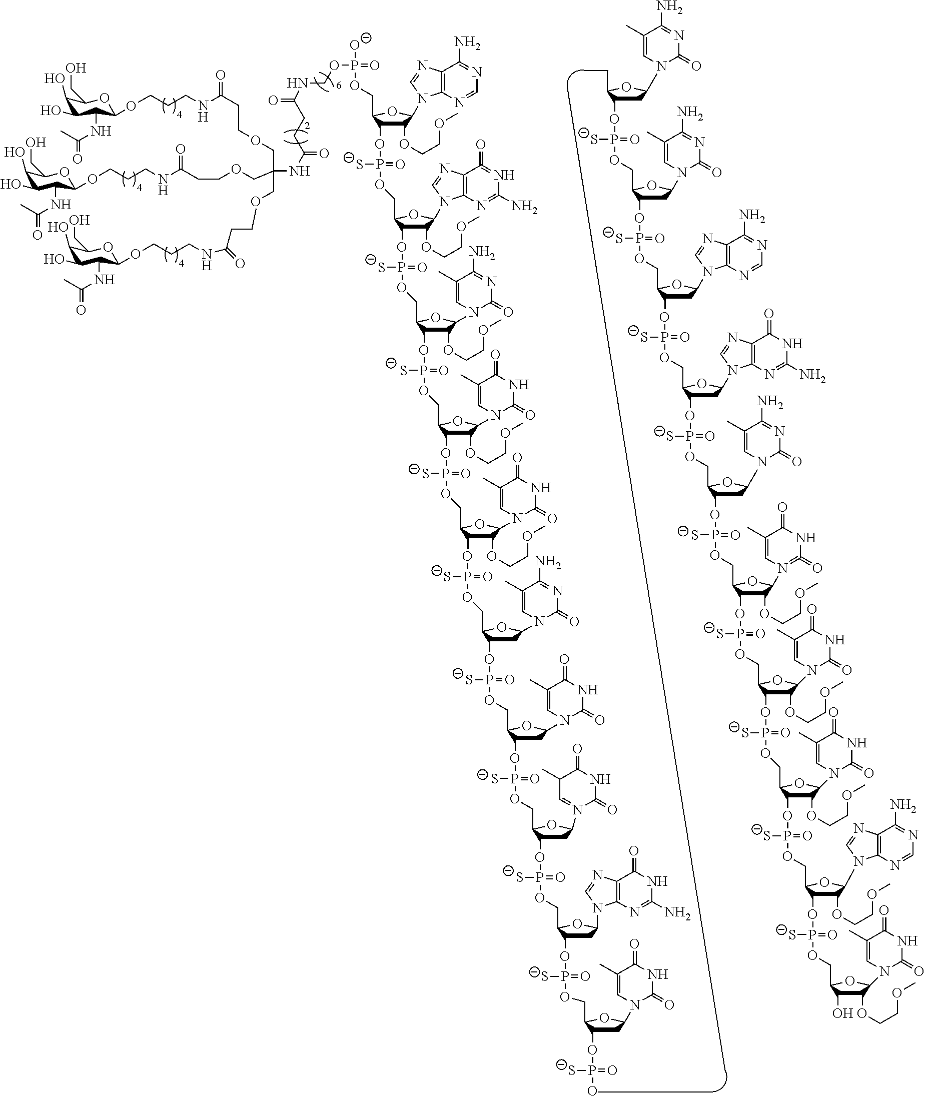

Certain embodiments provide use of a compound comprising an ApoCIII specific inhibitor for decreasing ApoCIII levels in an animal with Lipodystrophy. In certain embodiments, ApoCIII levels are decreased in the liver or small intestine.

Certain embodiments provide a compound comprising an ApoCIII specific inhibitor for use in: treating, preventing, delaying or ameliorating Partial Lipodystrophy, or a disease associated with Partial Lipodystophy in an animal; reducing triglyceride levels in an animal with Partial Lipodystrophy; increasing HDL levels and/or improving the ratio of TG to HDL in an animal with Partial Lipodystrophy; preventing, delaying or ameliorating a cardiovascular and/or metabolic disease, disorder, condition, or a symptom thereof, in an animal with Partial Lipodystrophy; and/or preventing, delaying or ameliorating pancreatitis, or a symptom thereof, in an animal with Partial Lipodystrophy.

Certain embodiments provide a compound comprising an ApoCIII specific inhibitor for use in the preparation of a medicament for treating, preventing, delaying or ameliorating Lipodystrophy.

Certain embodiments provide use of a compound comprising an ApoCIII specific inhibitor in the preparation of a medicament for decreasing ApoCIII levels in an animal with Lipodystrophy. In certain embodiments, ApoCIII levels are decreased in the liver or small intestine.

Certain embodiments provide a use of a compound comprising an ApoCIII specific inhibitor in the preparation of a medicament for decreasing TG levels, increasing HDL levels and/or improving the ratio of TG to HDL in an animal with Lipodystrophy.

Certain embodiments provide use of a compound comprising an ApoCIII specific inhibitor in the preparation of a medicament for preventing, treating, ameliorating or reducing at cardiovascular or metabolic disease in an animal with Lipodystrophy.

Certain embodiments provide use of a compound comprising an ApoCIII specific inhibitor in the preparation of a medicament for preventing, treating, ameliorating or reducing at pancreatitis in an animal with Lipodystrophy.

Certain embodiments provide use of a compound comprising an ApoCIII specific inhibitor in the preparation of a medicament for preventing, treating, ameliorating or reducing at hepatic steatosis, NAFLD, NASH, hepatic cirrhosis or hepatocarcinoma in an animal with Lipodystrophy.

In certain embodiments, the ApoCIII specific inhibitor used in the preparation of a medicament is a nucleic acid, peptide, antibody, small molecule or other agent capable of inhibiting the expression of ApoCIII. In certain embodiments, the nucleic acid is an antisense compound. In certain embodiments, the antisense compound is a modified oligonucleotide targeting ApoCIII. In certain embodiments, the modified oligonucleotide has a nucleobase sequence comprising at least 8 contiguous nucleobases of ISIS 304801 (SEQ ID NO: 3). In certain embodiments, the modified oligonucleotide is at least 70%, at least 75%, at least 80%, at least 85%, at least 90%, at least 95%, at least 98% or at least 100% complementary to SEQ ID NO: 1, SEQ ID NO: 2 or SEQ ID NO: 4.

In certain embodiments, the ApoCIII specific inhibitor used is a nucleic acid, peptide, antibody, small molecule or other agent capable of inhibiting the expression of ApoCIII. In certain embodiments, the nucleic acid is an antisense compound. In certain embodiments, the antisense compound is a modified oligonucleotide targeting ApoCIII. In certain embodiments, the modified oligonucleotide has a nucleobase sequence comprising at least 8 contiguous nucleobases of ISIS 304801 (SEQ ID NO: 3). In certain embodiments, the modified oligonucleotide is at least 70%, at least 75%, at least 80%, at least 85%, at least 90%, at least 95%, at least 98% or at least 100% complementary to SEQ ID NO: 1, SEQ ID NO: 2 or SEQ ID NO: 4.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that it is capable of undergoing hybridization to a target nucleic acid through hydrogen bonding.

Antisense compounds provided herein refer to oligomeric compounds capable of undergoing hybridization to a target nucleic acid through hydrogen bonding. Examples of antisense compounds include single-stranded and double-stranded compounds, such as, antisense oligonucleotides, siRNAs, shRNAs, and miRNAs.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to an ApoCIII nucleic acid is 12 to 30 nucleotides in length. In other words, antisense compounds are from 12 to 30 linked nucleobases. In other embodiments, the antisense compound comprises a modified oligonucleotide consisting of 8 to 80, 10 to 80, 12 to 50, 15 to 30, 18 to 24, 19 to 22, or 20 linked nucleobases. In certain such embodiments, the antisense compound comprises a modified oligonucleotide consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked nucleobases in length, or a range defined by any two of the above values. In some embodiments, the antisense compound is an antisense oligonucleotide.

In certain embodiments, the antisense compound comprises a shortened or truncated modified oligonucleotide. The shortened or truncated modified oligonucleotide can have one or more nucleosides deleted from the 5′ end (5′ truncation), one or more nucleosides deleted from the 3′ end (3′ truncation) or one or more nucleosides deleted from the central portion. Alternatively, the deleted nucleosides may be dispersed throughout the modified oligonucleotide, for example, in an antisense compound having one nucleoside deleted from the 5′ end and one nucleoside deleted from the 3′ end.

When a single additional nucleoside is present in a lengthened oligonucleotide, the additional nucleoside may be located at the central portion, 5′ or 3′ end of the oligonucleotide. When two or more additional nucleosides are present, the added nucleosides may be adjacent to each other, for example, in an oligonucleotide having two nucleosides added to the central portion, to the 5′ end (5′ addition), or alternatively to the 3′ end (3′ addition), of the oligonucleotide. Alternatively, the added nucleosides may be dispersed throughout the antisense compound, for example, in an oligonucleotide having one nucleoside added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to an ApoCIII nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of a RNA: DNA duplex.

Antisense compounds having a gapmer motif are considered chimeric antisense compounds. In a gapmer an internal region having a plurality of nucleotides that supports RNase H cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH2)n-O-2′ bridge, where n=1 or n=2). Preferably, each distinct region comprises uniform sugar moieties. The wing-gap-wing motif is frequently described as “X—Y—Z”, where “X” represents the length of the 5′ wing region, “Y” represents the length of the gap region, and “Z” represents the length of the 3′ wing region. As used herein, a gapmer described as “X—Y—Z” has a configuration such that the gap segment is positioned immediately adjacent to each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same; in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides. Thus, gapmers include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 6-8-6, 5-8-5, 1-8-1, 2-6-2, 2-13-2, 1-8-2, 2-8-3, 3-10-2, 1-18-2 or 2-18-2.

In certain embodiments, the antisense compound as a “wingmer” motif, having a wing-gap or gap-wing configuration, i.e. an X-Y or Y—Z configuration as described above for the gapmer configuration. Thus, wingmer configurations include, but are not limited to, for example 5-10, 8-4, 4-12, 12-4, 3-14, 16-2, 18-1, 10-3, 2-10, 1-10, 8-2, 2-13 or 5-13.

In certain embodiments, antisense compounds targeted to an ApoCIII nucleic acid possess a 5-10-5 gapmer motif.

In certain embodiments, an antisense compound targeted to an ApoCIII nucleic acid has a gap-widened motif.

### Target Nucleic Acids, Target Regions and Nucleotide Sequences

Nucleotide sequences that encode ApoCIII include, without limitation, the following: GENBANK Accession No. NM 000040.1 (incorporated herein as SEQ ID NO: 1), GENBANK Accession No. NT 033899.8 truncated from nucleotides 20262640 to 20266603 (incorporated herein as SEQ ID NO: 2) and GenBank Accession No. NT_035088.1 truncated from nucleotides 6238608 to U.S. Pat. No. 6,242,565 (incorporated herein as SEQ ID NO: 4).

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for ApoCIII can be obtained by accession number from sequence databases such as NCBI and such information is incorporated herein by reference. In certain embodiments, a target region may encompass the sequence from a 5′ target site of one target segment within the target region to a 3′ target site of another target segment within the target region.

In certain embodiments, a “target segment” is a smaller, sub-portion of a target region within a nucleic acid. For example, a target segment can be the sequence of nucleotides of a target nucleic acid to which one or more antisense compounds are targeted. “5′ target site” refers to the 5′-most nucleotide of a target segment. “3′ target site” refers to the 3′-most nucleotide of a target segment.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed, herein.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There can be variation in activity (e.g., as defined by percent reduction of target nucleic acid levels) of the antisense compounds within an active target region. In certain embodiments, reductions in ApoCIII mRNA levels are indicative of inhibition of ApoCIII expression. Reductions in levels of an ApoCIII protein can be indicative of inhibition of target mRNA expression. Further, phenotypic changes can be indicative of inhibition of ApoCIII expression. For example, an increase in HDL level, decrease in LDL level, or decrease in TG level are among phenotypic changes that may be assayed for inhibition of ApoCIII expression. Other phenotypic indications, e.g., symptoms associated with a cardiovascular or metabolic disease, may also be assessed; for example, angina; chest pain; shortness of breath; palpitations; weakness; dizziness; nausea; sweating; tachycardia; bradycardia; arrhythmia; atrial fibrillation; swelling in the lower extremities; cyanosis; fatigue; fainting; numbness of the face; numbness of the limbs; claudication or cramping of muscles; bloating of the abdomen; or fever.

### Hybridization

In some embodiments, hybridization occurs between an antisense compound disclosed herein and an ApoCIII nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art (Sambrook and Russell, Molecular Cloning: A Laboratory Manual, 3^{rd} Ed., 2001, CSHL Press). In certain embodiments, the antisense compounds provided herein are specifically hybridizable with an ApoCIII nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as an ApoCIII nucleic acid).

An antisense compound may hybridize over one or more segments of an ApoCIII nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 75%, 80%, 85%, 86%, 87%, 88%, 89%, 90%, 91%, 92%, 93%, 94%, 95%, 96%, 97%, 98%, 99%, or 100% complementary to an ApoCIII nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, an antisense compound which is 18 nucleobases in length having 4 (four) non-complementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

In certain embodiments, the antisense compounds provided herein, or specified portions thereof, are fully complementary (i.e. 100% complementary) to a target nucleic acid, or specified portion thereof. For example, an antisense compound may be fully complementary to an ApoCIII nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase(s) can be at the 5′ end or 3′ end of the antisense compound. Alternatively, the non-complementary nucleobase(s) can be at an internal position of the antisense compound. When two or more non-complementary nucleobases are present, they can be contiguous (i.e. linked) or non-contiguous. In one embodiment, a non-complementary nucleobase is located in the wing segment of a gapmer antisense oligonucleotide.

In certain embodiments, antisense compounds that are, or are up to, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as an ApoCIII nucleic acid, or specified portion thereof.

In certain embodiments, antisense compounds that are, or are up to, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as an ApoCIII nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds are complementary to at least an 8 nucleobase portion of a target segment. In certain embodiments, the antisense compounds are complementary to at least a 10 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or sequence of a compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides can also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to an ApoCIII nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds of the invention can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(Rₗ)—(CH₂)₂—N(Rₘ)(Rₙ), where each Rₗ, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleic acids (BNAs) include without limitation nucleosides comprising a bridge between the 4′ and the 2′ ribosyl ring atoms. In certain embodiments, antisense compounds provided herein include one or more BNA nucleosides wherein the bridge comprises one of the formulas: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see PCT/US2008/068922 published as WO/2009/006478, published Jan. 8, 2009); 4′-CH₂—N(OCH₃)-2′ (and analogs thereof see PCT/US2008/064591 published as WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof see PCT/US2008/066154 published as WO 2008/154401, published on Dec. 8, 2008).

Further bicyclic nucleosides have been reported in published literature (see for example: Srivastava et al., J. Am. Chem. Soc., 2007, 129(26) 8362-8379; Frieden et al., Nucleic Acids Research, 2003, 21, 6365-6372; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wahlestedt et al., Proc. Natl. Acad. Sci. U.S.A, 2000, 97, 5633-5638; Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; U.S. Pat. Nos. 7,399,845; 7,053,207; 7,034,133; 6,794,499; 6,770,748; 6,670,461; 6,525,191; 6,268,490; U.S. Patent Publication Nos.: US2008-0039618; US2007-0287831; US2004-0171570; U.S. patent application Ser. Nos. 12/129,154; 61/099,844; 61/097,787; 61/086,231; 61/056,564; 61/026,998; 61/026,995; 60/989,574; International applications WO 2007/134181; WO 2005/021570; WO 2004/106356; WO 94/14226; and PCT International Applications Nos.: PCT/US2008/068922; PCT/US2008/066154; and PCT/US2008/064591). Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

As used herein, “monocyclic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

In certain embodiments, bicyclic sugar moieties of BNA nucleosides include, but are not limited to, compounds having at least one bridge between the 4′ and the 2′ carbon atoms of the pentofuranosyl sugar moiety including without limitation, bridges comprising 1 or from 1 to 4 linked groups independently selected from —[C(Rₐ)(R_{b})]ₙ—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is, —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)- 2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-(CH₂)—O-2′ bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include those having a 4′ to 2′ bridge wherein such bridges include without limitation, α-L-4′-(CH₂)—O-2′, β-D-4′-CH₂—O-2′, 4′-(CH₂)₂—O-2′, 4′-CH₂—O—N(R)-2′, 4′-CH₂—N(R)—O-2′, 4′-CH(CH₃)—O-2′, 4′-CH₂—S-2′, 4′-CH₂—N(R)-2′, 4′-CH₂—CH(CH₃)-2′, and 4′-(CH₂)₃-2′, wherein R is H, a protecting group or C₁-C₁₂ alkyl.

In certain embodiment, bicyclic nucleosides have the formula:

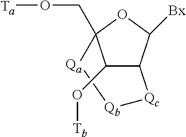

wherein:

Bx is a heterocyclic base moiety;

-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;

R_{c} is C₁-C₁₂ alkyl or an amino protecting group; and

Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides have the formula:

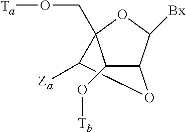

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thiol.

In one embodiment, each of the substituted groups, is, independently, mono or poly substituted with substituent groups independently selected from halogen, oxo, hydroxyl, OJ_{c}, NJ_{c}J_{d} SJ_{c}, N₃, OC(═X)J_{c}, and NJₑC(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides have the formula:

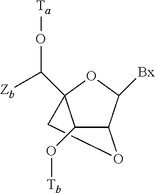

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides have the formula:

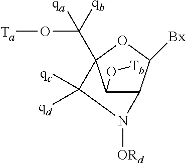

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides have the formula:

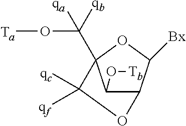

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(qₕ);

q_{g} and qₕ are each, independently, H, halogen, C₁-C₁₂ alkyl or substituted C₁-C₁₂ alkyl.

The synthesis and preparation of adenine, cytosine, guanine, 5-methyl-cytosine, thymine and uracil bicyclic nucleosides having a 4′-CH₂—O-2′ bridge, along with their oligomerization, and nucleic acid recognition properties have been described (Koshkin et al., Tetrahedron, 1998, 54, 3607-3630). The synthesis of bicyclic nucleosides has also been described in WO 98/39352 and WO 99/14226.

Analogs of various bicyclic nucleosides that have 4′ to 2′ bridging groups such as 4′-CH₂—O-2′ and 4′-CH₂—S-2′, have also been prepared (Kumar et al., Bioorg. Med. Chem. Lett., 1998, 8, 2219-2222). Preparation of oligodeoxyribonucleotide duplexes comprising bicyclic nucleosides for use as substrates for nucleic acid polymerases has also been described (Wengel et al., WO 99/14226). Furthermore, synthesis of 2′-amino-BNA, a novel conformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides have the formula:

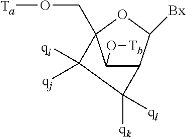

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Frier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc. 2007, 129(26), 8362-8379).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, (F) methyl(methyleneoxy) (4′-CH(CH₃)—O-2′) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA, and (K) vinyl BNA as depicted below.

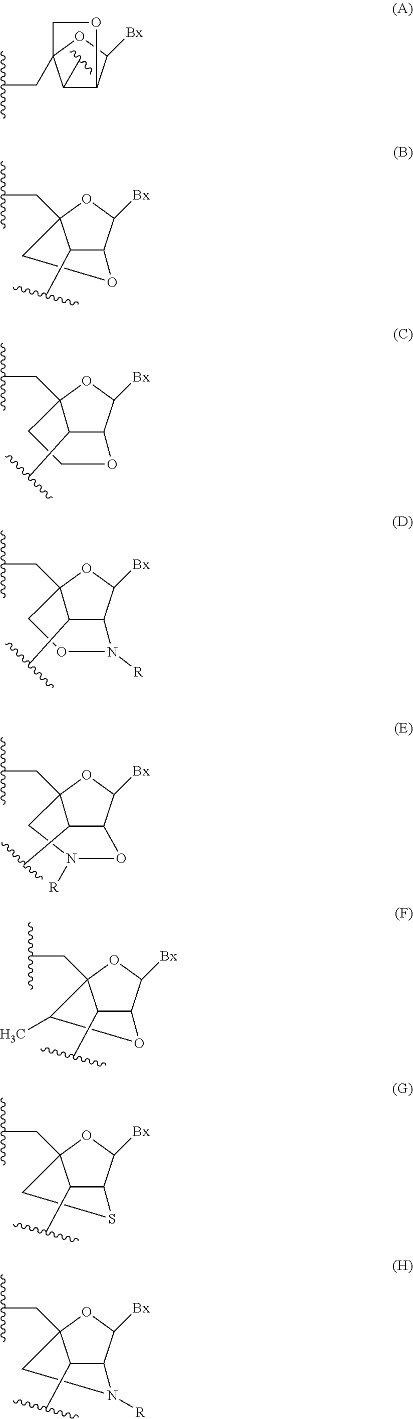

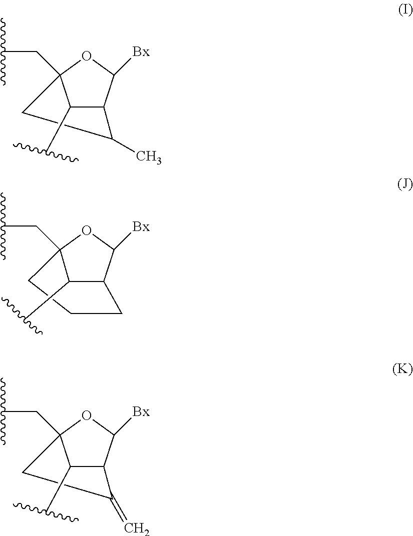

wherein Bx is the base moiety and R is, independently, H, a protecting group, C₁-C₆ alkyl or C₁-C₆ alkoxy.

As used herein, the term “modified tetrahydropyran nucleoside” or “modified THP nucleoside” means a nucleoside having a six-membered tetrahydropyran “sugar” substituted for the pentofuranosyl residue in normal nucleosides and can be referred to as a sugar surrogate. Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854) or fluoro HNA (F-HNA) having a tetrahydropyranyl ring system as illustrated below.

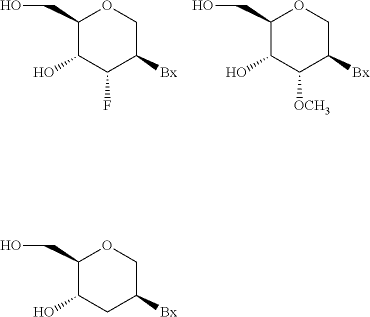

In certain embodiment, sugar surrogates are selected having the formula:

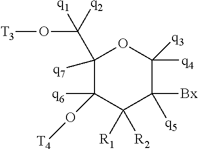

wherein:

Bx is a heterocyclic base moiety;

T₃ and T₄ are each, independently, an internucleoside linking group linking the tetrahydropyran nucleoside analog to the oligomeric compound or one of T₃ and T₄ is an internucleoside linking group linking the tetrahydropyran nucleoside analog to an oligomeric compound or oligonucleotide and the other of T₃ and T₄ is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and

one of R₁ and R₂ is hydrogen and the other is selected from halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides are provided wherein one of R₁ and R₂ is F. In certain embodiments, R₁ is fluoro and R₂ is H; R₁ is methoxy and R₂ is H, and R₁ is methoxyethoxy and R₂ is H.

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following formula:

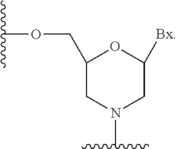

In certain embodiments, morpholinos may be modified, for example by adding or altering various substituent groups from the above morpholino structure. Such sugar surrogates are referred to herein as “modifed morpholinos.”

Combinations of modifications are also provided without limitation, such as 2′-F-5′-methyl substituted nucleosides (see PCT International Application WO 2008/101157 published on Aug. 21, 2008 for other disclosed 5′, 2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

In certain embodiments, antisense compounds comprise one or more modified cyclohexenyl nucleosides, which is a nucleoside having a six-membered cyclohexenyl in place of the pentofuranosyl residue in naturally occurring nucleosides. Modified cyclohexenyl nucleosides include, but are not limited to those described in the art (see for example commonly owned, published PCT Application WO 2010/036696, published on Apr. 10, 2010, Robeyns et al., J. Am. Chem. Soc., 2008, 130(6), 1979-1984; Horvath et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-998; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; Published PCT application, WO 06/047842; and Published PCT Application WO 01/049687; the text of each is incorporated by reference herein, in their entirety). Certain modified cyclohexenyl nucleosides have Formula X.

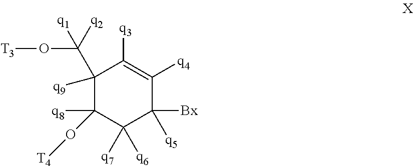

wherein independently for each of said at least one cyclohexenyl nucleoside analog of Formula X:

Bx is a heterocyclic base moiety;

T₃ and T₄ are each, independently, an internucleoside linking group linking the cyclohexenyl nucleoside analog to an antisense compound or one of T₃ and T₄ is an internucleoside linking group linking the tetrahydropyran nucleoside analog to an antisense compound and the other of T₃ and T₄ is H, a hydroxyl protecting group, a linked conjugate group, or a 5′- or 3′-terminal group; and

q₁, q₂, q₃, q₄, q₅, q₆, q₇, q₈ and q₉ are each, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or other sugar substituent group.

Many other monocyclic, bicyclic and tricyclic ring systems are known in the art and are suitable as sugar surrogates that can be used to modify nucleosides for incorporation into oligomeric compounds as provided herein (see for example review article: Leumann, Christian J. Bioorg. & Med. Chem., 2002, 10, 841-854). Such ring systems can undergo various additional substitutions to further enhance their activity.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modifed nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′ substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modifed nucleosides may further comprise other modifications, for example at other positions of the sugar and/or at the nucleobase.

As used herein, “2′-F” refers to a nucleoside comprising a sugar comprising a fluoro group at the 2′ position of the sugar ring.

As used herein, “2′-OMe” or “2′-OCH₃”, “2′-O-methyl” or “2′-methoxy” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

Methods for the preparations of modified sugars are well known to those skilled in the art. Some representative U.S. patents that teach the preparation of such modified sugars include without limitation, U.S.: 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,670,633; 5,700,920; 5,792,847 and 6,600,032 and International Application PCT/US2005/019219, filed Jun. 2, 2005 and published as WO 2005/121371 on Dec. 22, 2005, and each of which is herein incorporated by reference in its entirety.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)-0-2′) bridging group. In certain embodiments, the (4′-CH(CH₃)—O-2′) modified nucleosides are arranged throughout the wings of a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine.

Heterocyclic base moieties may include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and O-6 substituted purines, including 2 aminopropyladenine, 5-propynyluracil and 5-propynylcytosine.

In certain embodiments, antisense compounds targeted to an ApoCIII nucleic acid comprise one or more modified nucleobases. In certain embodiments, gap-widened antisense oligonucleotides targeted to an ApoCIII nucleic acid comprise one or more modified nucleobases. In certain embodiments, the modified nucleobase is 5-methylcytosine. In certain embodiments, each cytosine is a 5-methylcytosine.

### Certain Antisense Compound Motifs and Mechanisms

In certain embodiments, antisense compounds have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may confer another desired property e.g., serve as a substrate for the cellular endonuclease RNase H, which cleaves the RNA strand of an RNA:DNA duplex.

Antisense activity may result from any mechanism involving the hybridization of the antisense compound (e.g., oligonucleotide) with a target nucleic acid, wherein the hybridization ultimately results in a biological effect. In certain embodiments, the amount and/or activity of the target nucleic acid is modulated. In certain embodiments, the amount and/or activity of the target nucleic acid is reduced. In certain embodiments, hybridization of the antisense compound to the target nucleic acid ultimately results in target nucleic acid degradation. In certain embodiments, hybridization of the antisense compound to the target nucleic acid does not result in target nucleic acid degradation. In certain such embodiments, the presence of the antisense compound hybridized with the target nucleic acid (occupancy) results in a modulation of antisense activity. In certain embodiments, antisense compounds having a particular chemical motif or pattern of chemical modifications are particularly suited to exploit one or more mechanisms. In certain embodiments, antisense compounds function through more than one mechanism and/or through mechanisms that have not been elucidated. Accordingly, the antisense compounds described herein are not limited by particular mechanism.

Antisense mechanisms include, without limitation, RNase H mediated antisense; RNAi mechanisms, which utilize the RISC pathway and include, without limitation, siRNA, ssRNA and microRNA mechanisms; and occupancy based mechanisms. Certain antisense compounds may act through more than one such mechanism and/or through additional mechanisms.

RNase H-Mediated Antisense

In certain embodiments, antisense activity results at least in part from degradation of target RNA by RNase H. RNase H is a cellular endonuclease that cleaves the RNA strand of an RNA:DNA duplex. It is known in the art that single-stranded antisense compounds which are “DNA-like” elicit RNase H activity in mammalian cells. Accordingly, antisense compounds comprising at least a portion of DNA or DNA-like nucleosides may activate RNase H, resulting in cleavage of the target nucleic acid. In certain embodiments, antisense compounds that utilize RNase H comprise one or more modified nucleosides. In certain embodiments, such antisense compounds comprise at least one block of 1-8 modified nucleosides. In certain such embodiments, the modified nucleosides do not support RNase H activity. In certain embodiments, such antisense compounds are gapmers, as described herein. In certain such embodiments, the gap of the gapmer comprises DNA nucleosides. In certain such embodiments, the gap of the gapmer comprises DNA-like nucleosides. In certain such embodiments, the gap of the gapmer comprises DNA nucleosides and DNA-like nucleosides.

Certain antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE and 2′-O—CH₃, among others), and bicyclic sugar modified nucleosides (such bicyclic sugar modified nucleosides may include those having a constrained ethyl). In certain embodiments, nucleosides in the wings may include several modified sugar moieties, including, for example 2′-MOE and bicyclic sugar moieties such as constrained ethyl or LNA. In certain embodiments, wings may include several modified and unmodified sugar moieties. In certain embodiments, wings may include various combinations of 2′-MOE nucleosides, bicyclic sugar moieties such as constrained ethyl nucleosides or LNA nucleosides, and 2′-deoxynucleosides.

Each distinct region may comprise uniform sugar moieties, variant, or alternating sugar moieties. The wing-gap-wing motif is frequently described as “X—Y—Z”, where “X” represents the length of the 5′-wing, “Y” represents the length of the gap, and “Z” represents the length of the 3′-wing. “X” and “Z” may comprise uniform, variant, or alternating sugar moieties. In certain embodiments, “X” and “Y” may include one or more 2′-deoxynucleosides. “Y” may comprise 2′-deoxynucleosides. As used herein, a gapmer described as “X—Y—Z” has a configuration such that the gap is positioned immediately adjacent to each of the 5′-wing and the 3′ wing. Thus, no intervening nucleotides exist between the 5′-wing and gap, or the gap and the 3′-wing. Any of the antisense compounds described herein can have a gapmer motif. In certain embodiments, “X” and “Z” are the same; in other embodiments they are different. In certain embodiments, “Y” is between 8 and 15 nucleosides. X, Y, or Z can be any of 1, 2, 3, 4, 5, 6, 7, 8, 9, 10, 11, 12, 13, 14, 15, 16, 17, 18, 19, 20, 25, 30 or more nucleosides.

In certain embodiments, the antisense compound targeted to an APOCIII nucleic acid has a gapmer motif in which the gap consists of 6, 7, 8, 9, 10, 11, 12, 13, 14, 15, or 16 linked nucleosides.

In certain embodiments, the antisense oligonucleotide has a sugar motif described by Formula A as follows: (J)ₘ-(B)ₙ-(J)ₚ-(B)ᵣ-(A)ₜ-(D)_{g}-(A)ᵥ-(B)_{w}-(J)ₓ-(B)_{y}-(J)_{z}

wherein:
each A is independently a 2′-substituted nucleoside;
each B is independently a bicyclic nucleoside;
each J is independently either a 2′-substituted nucleoside or a 2′-deoxynucleoside; each D is a 2′-deoxynucleoside;
m is 0-4; n is 0-2; p is 0-2; r is 0-2; t is 0-2; v is 0-2; w is 0-4; x is 0-2; y is 0-2; z is 0-4; g is 6-14;
   provided that:
at least one of m, n, and r is other than 0;
at least one of w and y is other than 0;
the sum of m, n, p, r, and t is from 2 to 5; and
the sum of v, w, x, y, and z is from 2 to 5.

RNAi Compounds

In certain embodiments, antisense compounds are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). In certain embodiments, antisense compounds comprise modifications that make them particularly suited for such mechanisms.

i. ssRNA Compounds

In certain embodiments, antisense compounds including those particularly suited for use as single-stranded RNAi compounds (ssRNA) comprise a modified 5′-terminal end. In certain such embodiments, the 5′-terminal end comprises a modified phosphate moiety. In certain embodiments, such modified phosphate is stabilized (e.g., resistant to degradation/cleavage compared to unmodified 5′-phosphate). In certain embodiments, such 5′-terminal nucleosides stabilize the 5′-phosphorous moiety. Certain modified 5′-terminal nucleosides may be found in the art, for example in WO/2011/139702.

In certain embodiments, the 5′-nucleoside of an ssRNA compound has Formula IIc:

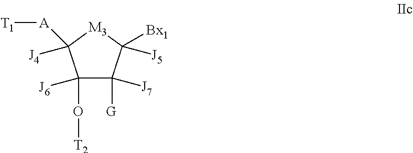

wherein:

T₁ is an optionally protected phosphorus moiety;

T₂ is an internucleoside linking group linking the compound of Formula IIc to the oligomeric compound;

A has one of the formulas:

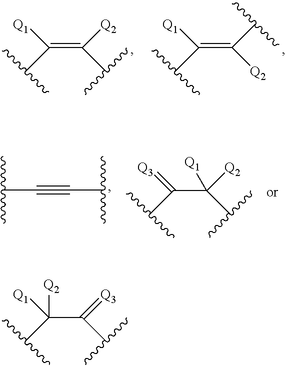

Q₁ and Q₂ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, substituted C₂-C₆ alkynyl or N(R₃)(R₄);

Q₃ is O, S, N(R₅) or C(R₆)(R₇);

each R₃, R₄ R₅, R₆ and R₇ is, independently, H, C₁-C₆ alkyl, substituted C₁-C₆ alkyl or C₁-C₆ alkoxy;

M₃ is O, S, NR₁₄, C(R₁₅)(R₁₆), C(R₁₅)(R₁₆)C(R₁₇)(R₁₈), C(R₁₅)═C(R₁₇), OC(R₁₅)(R₁₆) or OC(R₁₅)(Bx₂);

R₁₄ is H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;

R₁₅, R₁₆, R₁₇ and R₁₈ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;

Bx₁ is a heterocyclic base moiety;

or if Bx₂ is present then Bx₂ is a heterocyclic base moiety and Bx₁ is H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;

J₄, J₅, J₆ and J₇ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;

or J₄ forms a bridge with one of J₅ or J₇ wherein said bridge comprises from 1 to 3 linked biradical groups selected from O, S, NR₁₉, C(R₂₀)(R₂₁), C(R₂₀)═C(R₂₁), C[═C(R₂₀)(R₂₁)] and C(═O) and the other two of J₅, J₆ and J₇ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;

each R₁₉, R₂₀ and R₂₁ is, independently, H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;

G is H, OH, halogen or O—[C(R₈)(R₉)]ₙ—[(C═O)ₘ—X₁]ⱼ—Z;

each R₈ and R₉ is, independently, H, halogen, C₁-C₆ alkyl or substituted C₁-C₆ alkyl;

X₁ is O, S or N(E₁);

Z is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);

E₁, E₂ and E₃ are each, independently, H, C₁-C₆ alkyl or substituted C₁-C₆ alkyl;

n is from 1 to about 6;

m is 0 or 1;

j is 0 or 1;

each substituted group comprises one or more optionally protected substituent groups independently selected from halogen, OJ₁, N(J₁)(J₂), =NJ₁, SJ₁, N₃, CN, OC(═X₂)J₁, OC(═X₂)—N(J₁)(J₂) and C(═X₂)N(J₁)(J₂);

X₂ is O, S or NJ₃;

each J₁, J₂ and J₃ is, independently, H or C₁-C₆ alkyl;

when j is 1 then Z is other than halogen or N(E₂)(E₃); and

wherein said oligomeric compound comprises from 8 to 40 monomeric subunits and is hybridizable to at least a portion of a target nucleic acid.

In certain embodiments, M₃ is O, CH═CH, OCH₂ or OC(H)(Bx₂). In certain embodiments, M₃ is O.

In certain embodiments, J₄, J₅, J₆ and J₇ are each H. In certain embodiments, J₄ forms a bridge with one of J₅ or J₇.

In certain embodiments, A has one of the formulas:

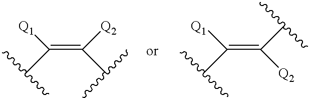

wherein:

Q₁ and Q₂ are each, independently, H, halogen, C₁-C₆ alkyl, substituted C₁-C₆ alkyl, C₁-C₆ alkoxy or substituted C₁-C₆ alkoxy. In certain embodiments, Q₁ and Q₂ are each H. In certain embodiments, Q₁ and Q₂ are each, independently, H or halogen. In certain embodiments, Q₁ and Q₂ is H and the other of Q₁ and Q₂ is F, CH₃ or OCH₃.

In certain embodiments, T₁ has the formula:

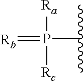

wherein:

Rₐ and R_{c} are each, independently, protected hydroxyl, protected thiol, C₁-C₆ alkyl, substituted C₁-C₆ alkyl, C₁-C₆ alkoxy, substituted C₁-C₆ alkoxy, protected amino or substituted amino; and

R_{b} is O or S. In certain embodiments, R_{b} is O and Rₐ and R_{c} are each, independently, OCH₃, OCH₂CH₃ or CH(CH₃)₂.

In certain embodiments, G is halogen, OCH₃, OCH₂F, OCHF₂, OCF₃, OCH₂CH₃, O(CH₂)₂F, OCH₂CHF₂, OCH₂CF₃, OCH₂—CH═CH₂, O(CH₂)₂—OCH₃, O(CH₂)₂—SCH₃, O(CH₂)₂—OCF₃, O(CH₂)₃—N(R₁₀)(R₁₁), O(CH₂)₂—ON(R₁₀)(R₁₁), O(CH₂)₂—O(CH₂)₂—N(R₁₀)(R₁₁), OCH₂C(═O)—N(R₁₀)(R₁₁), OCH₂C(═O)—N(R₁₂)—(CH₂)₂—N(R₁₀)(R₁₁) or O(CH₂)₂—N(R₁₂)—C(═NR₁₃)[N(R₁₀)(R₁₁)] wherein R₁₀, R₁₁, R₁₂ and R₁₃ are each, independently, H or C₁-C₆ alkyl. In certain embodiments, G is halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂ or OCH₂—N(H)—C(═NH)NH₂. In certain embodiments, G is F, OCH₃ or O(CH₂)₂—OCH₃. In certain embodiments, G is O(CH₂)₂—OCH₃.

In certain embodiments, the 5′-terminal nucleoside has Formula He:

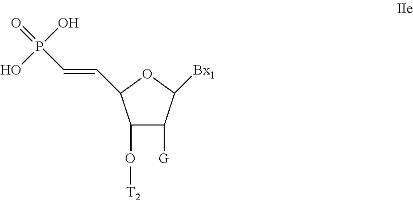

In certain embodiments, antisense compounds, including those particularly suitable for ssRNA comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having uniform sugar modifications. In certain such embodiments, each nucleoside of the region comprises the same RNA-like sugar modification. In certain embodiments, each nucleoside of the region is a 2′-F nucleoside. In certain embodiments, each nucleoside of the region is a 2′-OMe nucleoside. In certain embodiments, each nucleoside of the region is a 2′-MOE nucleoside. In certain embodiments, each nucleoside of the region is a cEt nucleoside. In certain embodiments, each nucleoside of the region is an LNA nucleoside. In certain embodiments, the uniform region constitutes all or essentially all of the oligonucleotide. In certain embodiments, the region constitutes the entire oligonucleotide except for 1-4 terminal nucleosides.

In certain embodiments, oligonucleotides comprise one or more regions of alternating sugar modifications, wherein the nucleosides alternate between nucleotides having a sugar modification of a first type and nucleotides having a sugar modification of a second type. In certain embodiments, nucleosides of both types are RNA-like nucleosides. In certain embodiments the alternating nucleosides are selected from: 2′-OMe, 2′-F, 2′-MOE, LNA, and cEt. In certain embodiments, the alternating modificatios are 2′-F and 2′-OMe. Such regions may be contiguous or may be interupted by differently modified nucleosides or conjugated nucleosides.

In certain embodiments, the alternating region of alternating modifications each consist of a single nucleoside (i.e., the patern is (AB)ₓA_{y} wheren A is a nucleoside having a sugar modification of a first type and B is a nucleoside having a sugar modification of a second type; x is 1-20 and y is 0 or 1). In certan embodiments, one or more alternating regions in an alternating motif includes more than a single nucleoside of a type. For example, oligonucleotides may include one or more regions of any of the following nucleoside motifs:

### AABBAA;

### ABBABB;

### AABAAB;

### ABBABAABB;

### ABABAA;

### AABABAB;

### ABABAA;

### ABBAABBABABAA;

### BABBAABBABABAA; or

### ABABBAABBABABAA;

wherein A is a nucleoside of a first type and B is a nucleoside of a second type. In certain embodiments, A and B are each selected from 2′-F, 2′-OMe, BNA, and MOE.

In certain embodiments, oligonucleotides having such an alternating motif also comprise a modified 5′ terminal nucleoside, such as those of formula IIc or IIe.

In certain embodiments, oligonucleotides comprise a region having a 2-2-3 motif. Such regions comprises the following motif:

-(A)₂-(B)ₓ(A)₂-(C)_{y}-(A)₃-

wherein: A is a first type of modifed nucleosde;

B and C, are nucleosides that are differently modified than A, however, B and C may have the same or different modifications as one another;

x and y are from 1 to 15.

In certain embodiments, A is a 2′-OMe modified nucleoside. In certain embodiments, B and C are both 2′-F modified nucleosides. In certain embodiments, A is a 2′-OMe modified nucleoside and B and C are both 2′-F modified nucleosides.

In certain embodiments, oligonucleosides have the following sugar motif:

5′-(Q)-(AB)ₓA_{y}-(D)_{z}

wherein:

Q is a nucleoside comprising a stabilized phosphate moiety. In certain embodiments, Q is a nucleoside having Formula IIc or IIe;

A is a first type of modifed nucleoside;

B is a second type of modified nucleoside;

D is a modified nucleoside comprising a modification different from the nucleoside adjacent to it. Thus, if y is 0, then D must be differently modified than B and if y is 1, then D must be differently modified than A. In certain embodiments, D differs from both A and B.

X is 5-15;

Y is 0 or 1;

Z is 0-4.

In certain embodiments, oligonucleosides have the following sugar motif:

5′-(Q)-(A)ₓ-(D)_{z}

wherein:

Q is a nucleoside comprising a stabilized phosphate moiety. In certain embodiments, Q is a nucleoside having Formula IIc or IIe;

A is a first type of modifed nucleoside;

D is a modified nucleoside comprising a modification different from A.

X is 11-30;

Z is 0-4.

In certain embodiments A, B, C, and D in the above motifs are selected from: 2′-OMe, 2′-F, 2′-MOE, LNA, and cEt. In certain embodiments, D represents terminal nucleosides. In certain embodiments, such terminal nucleosides are not designed to hybridize to the target nucleic acid (though one or more might hybridize by chance). In certiain embodiments, the nucleobase of each D nucleoside is adenine, regardless of the identity of the nucleobase at the corresponding position of the target nucleic acid. In certain embodiments the nucleobase of each D nucleoside is thymine.

In certain embodiments, antisense compounds, including those particularly suited for use as ssRNA comprise modified internucleoside linkages arranged along the oligonucleotide or region thereof in a defined pattern or modified internucleoside linkage motif. In certain embodiments, oligonucleotides comprise a region having an alternating internucleoside linkage motif. In certain embodiments, oligonucleotides comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate internucleoside linkages. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least one block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

Oligonucleotides having any of the various sugar motifs described herein, may have any linkage motif. For example, the oligonucleotides, including but not limited to those described above, may have a linkage motif selected from non-limiting the table below:

| 5′ most linkage | Central region | 3′-region |
|---|---|---|
| PS | Alternating PO/PS | 6 PS |
| PS | Alternating PO/PS | 7 PS |
| PS | Alternating PO/PS | 8 PS |

ii. siRNA Compounds

In certain embodiments, antisense compounds are double-stranded RNAi compounds (siRNA). In such embodiments, one or both strands may comprise any modification motif described above for ssRNA. In certain embodiments, ssRNA compounds may be unmodified RNA. In certain embodiments, siRNA compounds may comprise unmodified RNA nucleosides, but modified internucleoside linkages.

Several embodiments relate to double-stranded compositions wherein each strand comprises a motif defined by the location of one or more modified or unmodified nucleosides. In certain embodiments, compositions are provided comprising a first and a second oligomeric compound that are fully or at least partially hybridized to form a duplex region and further comprising a region that is complementary to and hybridizes to a nucleic acid target. It is suitable that such a composition comprise a first oligomeric compound that is an antisense strand having full or partial complementarity to a nucleic acid target and a second oligomeric compound that is a sense strand having one or more regions of complementarity to and forming at least one duplex region with the first oligomeric compound.

The compositions of several embodiments modulate gene expression by hybridizing to a nucleic acid target resulting in loss of its normal function. In some embodiments, the target nucleic acid is APOCIII. In certain embodiment, the degradation of the targeted APOCIII is facilitated by an activated RISC complex that is formed with compositions disclosed herein.

Several embodiments are directed to double-stranded compositions wherein one of the strands is useful in, for example, influencing the preferential loading of the opposite strand into the RISC (or cleavage) complex. The compositions are useful for targeting selected nucleic acid molecules and modulating the expression of one or more genes. In some embodiments, the compositions of the present invention hybridize to a portion of a target RNA resulting in loss of normal function of the target RNA.

Certain embodiments are drawn to double-stranded compositions wherein both the strands comprises a hemimer motif, a fully modified motif, a positionally modified motif or an alternating motif. Each strand of the compositions of the present invention can be modified to fulfil a particular role in for example the siRNA pathway. Using a different motif in each strand or the same motif with different chemical modifications in each strand permits targeting the antisense strand for the RISC complex while inhibiting the incorporation of the sense strand. Within this model, each strand can be independently modified such that it is enhanced for its particular role. The antisense strand can be modified at the 5′-end to enhance its role in one region of the RISC while the 3′-end can be modified differentially to enhance its role in a different region of the RISC.

The double-stranded oligonucleotide molecules can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The double-stranded oligonucleotide molecules can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double-stranded structure, for example wherein the double-stranded region is about 15 to about 30, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the double-stranded oligonucleotide molecule are complementary to the target nucleic acid or a portion thereof). Alternatively, the double-stranded oligonucleotide is assembled from a single oligonucleotide, where the self-complementary sense and antisense regions of the siRNA are linked by means of a nucleic acid based or non-nucleic acid-based linker(s).

The double-stranded oligonucleotide can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The double-stranded oligonucleotide can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNAi.

In certain embodiments, the double-stranded oligonucleotide comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the double-stranded oligonucleotide comprises nucleotide sequence that is complementary to nucleotide sequence of a target gene. In another embodiment, the double-stranded oligonucleotide interacts with nucleotide sequence of a target gene in a manner that causes inhibition of expression of the target gene.

As used herein, double-stranded oligonucleotides need not be limited to those molecules containing only RNA, but further encompasses chemically modified nucleotides and non-nucleotides. In certain embodiments, the short interfering nucleic acid molecules lack 2′-hydroxy (2′-OH) containing nucleotides. In certain embodiments short interfering nucleic acids optionally do not include any ribonucleotides (e.g., nucleotides having a 2′-OH group). Such double-stranded oligonucleotides that do not require the presence of ribonucleotides within the molecule to support RNAi can however have an attached linker or linkers or other attached or associated groups, moieties, or chains containing one or more nucleotides with 2′-OH groups. Optionally, double-stranded oligonucleotides can comprise ribonucleotides at about 5, 10, 20, 30, 40, or 50% of the nucleotide positions. As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, double-stranded oligonucleotides can be used to epigenetically silence genes at both the post-transcriptional level and the pre-transcriptional level. In a non-limiting example, epigenetic regulation of gene expression by siRNA molecules of the invention can result from siRNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

It is contemplated that compounds and compositions of several embodiments provided herein can target APOCIII by a dsRNA-mediated gene silencing or RNAi mechanism, including, e.g., “hairpin” or stem-loop double-stranded RNA effector molecules in which a single RNA strand with self-complementary sequences is capable of assuming a double-stranded conformation, or duplex dsRNA effector molecules comprising two separate strands of RNA. In various embodiments, the dsRNA consists entirely of ribonucleotides or consists of a mixture of ribonucleotides and deoxynucleotides, such as the RNA/DNA hybrids disclosed, for example, by WO 00/63364, filed Apr. 19, 2000, or U.S. Ser. No. 60/130,377, filed Apr. 21, 1999. The dsRNA or dsRNA effector molecule may be a single molecule with a region of self-complementarity such that nucleotides in one segment of the molecule base pair with nucleotides in another segment of the molecule. In various embodiments, a dsRNA that consists of a single molecule consists entirely of ribonucleotides or includes a region of ribonucleotides that is complementary to a region of deoxyribonucleotides. Alternatively, the dsRNA may include two different strands that have a region of complementarity to each other.

In various embodiments, both strands consist entirely of ribonucleotides, one strand consists entirely of ribonucleotides and one strand consists entirely of deoxyribonucleotides, or one or both strands contain a mixture of ribonucleotides and deoxyribonucleotides. In certain embodiments, the regions of complementarity are at least 70, 80, 90, 95, 98, or 100% complementary to each other and to a target nucleic acid sequence. In certain embodiments, the region of the dsRNA that is present in a double-stranded conformation includes at least 19, 20, 21, 22, 23, 24, 25, 26, 27, 28, 29, 30, 50, 75, 100, 200, 500, 1000, 2000 or 5000 nucleotides or includes all of the nucleotides in a cDNA or other target nucleic acid sequence being represented in the dsRNA. In some embodiments, the dsRNA does not contain any single stranded regions, such as single stranded ends, or the dsRNA is a hairpin. In other embodiments, the dsRNA has one or more single stranded regions or overhangs. In certain embodiments, RNA/DNA hybrids include a DNA strand or region that is an antisense strand or region (e.g, has at least 70, 80, 90, 95, 98, or 100% complementarity to a target nucleic acid) and an RNA strand or region that is a sense strand or region (e.g, has at least 70, 80, 90, 95, 98, or 100% identity to a target nucleic acid), and vice versa.

In various embodiments, the RNA/DNA hybrid is made in vitro using enzymatic or chemical synthetic methods such as those described herein or those described in WO 00/63364, filed Apr. 19, 2000, or U.S. Ser. No. 60/130,377, filed Apr. 21, 1999. In other embodiments, a DNA strand synthesized in vitro is complexed with an RNA strand made in vivo or in vitro before, after, or concurrent with the transformation of the DNA strand into the cell. In yet other embodiments, the dsRNA is a single circular nucleic acid containing a sense and an antisense region, or the dsRNA includes a circular nucleic acid and either a second circular nucleic acid or a linear nucleic acid (see, for example, WO 00/63364, filed Apr. 19, 2000, or U.S. Ser. No. 60/130,377, filed Apr. 21, 1999.) Exemplary circular nucleic acids include lariat structures in which the free 5′ phosphoryl group of a nucleotide becomes linked to the 2′ hydroxyl group of another nucleotide in a loop back fashion.

In other embodiments, the dsRNA includes one or more modified nucleotides in which the 2′ position in the sugar contains a halogen (such as fluorine group) or contains an alkoxy group (such as a methoxy group) which increases the half-life of the dsRNA in vitro or in vivo compared to the corresponding dsRNA in which the corresponding 2′ position contains a hydrogen or an hydroxyl group. In yet other embodiments, the dsRNA includes one or more linkages between adjacent nucleotides other than a naturally-occurring phosphodiester linkage. Examples of such linkages include phosphoramide, phosphorothioate, and phosphorodithioate linkages. The dsRNAs may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000, or U.S. Ser. No. 60/130,377, filed Apr. 21, 1999.

In other embodiments, the dsRNA can be any of the at least partially dsRNA molecules disclosed in WO 00/63364, as well as any of the dsRNA molecules described in U.S. Provisional Application 60/399,998; and U.S. Provisional Application 60/419,532, and PCT/US2003/033466, the teaching of which is hereby incorporated by reference. Any of the dsRNAs may be expressed in vitro or in vivo using the methods described herein or standard methods, such as those described in WO 00/63364.

### Occupancy

In certain embodiments, antisense compounds are not expected to result in cleavage or the target nucleic acid via RNase H or to result in cleavage or sequestration through the RISC pathway. In certain such embodiments, antisense activity may result from occupancy, wherein the presence of the hybridized antisense compound disrupts the activity of the target nucleic acid. In certain such embodiments, the antisense compound may be uniformly modified or may comprise a mix of modifications and/or modified and unmodified nucleosides.

### Compositions and Methods for Formulating Pharmaceutical Compositions

Antisense compounds may be admixed with pharmaceutically acceptable active or inert substance for the preparation of pharmaceutical compositions or formulations. Compositions and methods for the formulation of pharmaceutical compositions are dependent upon a number of criteria, including, but not limited to, route of administration, extent of disease, or dose to be administered.

Antisense compounds targeted to an ApoCIII nucleic acid can be utilized in pharmaceutical compositions by combining the antisense compound with a suitable pharmaceutically acceptable diluent or carrier. In certain embodiments, the “pharmaceutical carrier” or “excipient” is a pharmaceutically acceptable solvent, suspending agent or any other pharmacologically inert vehicle for delivering one or more nucleic acids to an animal. The excipient can be liquid or solid and can be selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients, which do not deleteriously react with nucleic acids, suitable for parenteral or non-parenteral administration can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to an ApoCIII nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or an oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds can be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes. In certain embodiments, the conjugate comprises a carbohydrate. In certain embodiments, the conjugate group comprises one or more N-Acetylgalactosamine (or “GalNAC”) moieties. In certain embodiments, the conjugate group comprises one, two, or three N-Acetylgalactosamine (or “GalNAc”) moieties.

Representative United States patents, United States patent application publications, and international patent application publications that teach the preparation of certain GalNAc conjugates, conjugated oligomeric compounds such as antisense compounds, tethers, conjugate linkers, branching groups, ligands, cleavable moieties as well as other modifications include without limitation, U.S. Pat. No. 5,994,517, U.S. Pat. No. 6,300,319, U.S. Pat. No. 6,660,720, U.S. Pat. No. 6,906,182, U.S. Pat. No. 7,262,177, U.S. Pat. No. 7,491,805, U.S. Pat. No. 8,106,022, U.S. Pat. No. 7,723,509, US 2006/0148740, US 2011/0123520, WO 2013/033230, WO 2012/037254, and WO 2014022739, each of which is incorporated by reference herein in its entirety.

Representative publications that teach the preparation of certain of GalNAc conjugates, conjugated oligomeric compounds such as antisense compounds, tethers, conjugate linkers, branching groups, ligands, cleavable moieties as well as other modifications include without limitation, BIESSEN et al., “The Cholesterol Derivative of a Triantennary Galactoside with High Affinity for the Hepatic Asialoglycoprotein Receptor: a Potent Cholesterol Lowering Agent” J. Med. Chem. (1995) 38:1846-1852, BIESSEN et al., “Synthesis of Cluster Galactosides with High Affinity for the Hepatic Asialoglycoprotein Receptor” J. Med. Chem. (1995) 38:1538-1546, LEE et al., “New and more efficient multivalent glyco-ligands for asialoglycoprotein receptor of mammalian hepatocytes” Bioorganic & Medicinal Chemistry (2011) 19:2494-2500, RENSEN et al., “Determination of the Upper Size Limit for Uptake and Processing of Ligands by the Asialoglycoprotein Receptor on Hepatocytes in Vitro and in Vivo” J. Biol. Chem. (2001) 276(40):37577-37584, RENSEN et al., “Design and Synthesis of Novel N-Acetylgalactosamine-Terminated Glycolipids for Targeting of Lipoproteins to the Hepatic Asialoglycoprotein Receptor” J. Med. Chem. (2004) 47:5798-5808, SLIEDREGT et al., “Design and Synthesis of Novel Amphiphilic Dendritic Galactosides for Selective Targeting of Liposomes to the Hepatic Asialoglycoprotein Receptor” J. Med. Chem. (1999) 42:609-618, R. T. Lee et al., “New and more efficient multivalent glyco-ligands for asialoglycoprote in receptor of mammalian hepatocytes,” Bioorg. Med. Chem. 19 (2011) 2494-2500, and Valentijn et al., “Solid-phase synthesis of lysine-based cluster galactosides with high affinity for the Asialoglycoprotein Receptor” Tetrahedron, 1997, 53(2), 759-770, each of which is incorporated by reference herein in its entirety.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acids from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of ApoCIII nucleic acids or proteins can be tested in vitro in a variety of cell types. Cell types used for such analyses are available from commercial vendors (e.g. American Type Culture Collection, Manassas, Va.; Zen-Bio, Inc., Research Triangle Park, N.C.; Clonetics Corporation, Walkersville, Md.) and cells are cultured according to the vendor's instructions using commercially available reagents (e.g. Invitrogen Life Technologies, Carlsbad, Calif.). Illustrative cell types include, but are not limited to, HepG2 cells, Hep3B cells, Huh7 (hepatocellular carcinoma) cells, primary hepatocytes, A549 cells, GM04281 fibroblasts and LLC-MK2 cells.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluence in culture.

One reagent commonly used to introduce antisense oligonucleotides into cultured cells includes the cationic lipid transfection reagent LIPOFECTIN® (Invitrogen, Carlsbad, Calif.). Antisense oligonucleotides are mixed with LIPOFECTIN® in OPTI-MEM® 1 (Invitrogen, Carlsbad, Calif.) to achieve the desired final concentration of antisense oligonucleotide and a LIPOFECTIN® concentration that typically ranges 2 to 12 ug/mL per 100 nM antisense oligonucleotide.

Another reagent used to introduce antisense oligonucleotides into cultured cells includes LIPOFECTAMINE 2000® (Invitrogen, Carlsbad, Calif.). Antisense oligonucleotide is mixed with LIPOFECTAMINE 2000® in OPTI-MEM® 1 reduced serum medium (Invitrogen, Carlsbad, Calif.) to achieve the desired concentration of antisense oligonucleotide and a LIPOFECTAMINE® concentration that typically ranges 2 to 12 ug/mL per 100 nM antisense oligonucleotide.

Another reagent used to introduce antisense oligonucleotides into cultured cells includes Cytofectin® (Invitrogen, Carlsbad, Calif.). Antisense oligonucleotide is mixed with Cytofectin® in OPTI-MEM® 1 reduced serum medium (Invitrogen, Carlsbad, Calif.) to achieve the desired concentration of antisense oligonucleotide and a Cytofectin® concentration that typically ranges 2 to 12 ug/mL per 100 nM antisense oligonucleotide.

Another reagent used to introduce antisense oligonucleotides into cultured cells includes Oligofectamine™ (Invitrogen Life Technologies, Carlsbad, Calif.). Antisense oligonucleotide is mixed with Oligofectamine™ in Opti-MEM™-1 reduced serum medium (Invitrogen Life Technologies, Carlsbad, Calif.) to achieve the desired concentration of oligonucleotide with an Oligofectamine™ to oligonucleotide ratio of approximately 0.2 to 0.8 μL per 100 nM.

Another reagent used to introduce antisense oligonucleotides into cultured cells includes FuGENE 6 (Roche Diagnostics Corp., Indianapolis, Ind.). Antisense oligomeric compound was mixed with FuGENE 6 in 1 mL of serum-free RPMI to achieve the desired concentration of oligonucleotide with a FuGENE 6 to oligomeric compound ratio of 1 to 4 μL of FuGENE 6 per 100 nM.

Another technique used to introduce antisense oligonucleotides into cultured cells includes electroporation (Sambrook and Russell in Molecular Cloning. A Laboratory Manual. Third Edition. Cold Spring Harbor Laboratory Press, Cold Spring Harbor, N.Y. 2001).

Cells are treated with antisense oligonucleotides by routine methods. Cells are typically harvested 16-24 hours after antisense oligonucleotide treatment, at which time RNA or protein levels of target nucleic acids are measured by methods known in the art and described herein (Sambrook and Russell in Molecular Cloning. A Laboratory Manual. Third Edition. Cold Spring Harbor Laboratory Press, Cold Spring Harbor, N.Y. 2001). In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art (Sambrook and Russell in Molecular Cloning. A Laboratory Manual. Third Edition. Cold Spring Harbor Laboratory Press, Cold Spring Harbor, N.Y. 2001). Antisense oligonucleotides are typically used at concentrations ranging from 1 nM to 300 nM when transfected with LIPOFECTAMINE2000® (Invitrogen, Carlsbad, Calif.), Lipofectin® (Invitrogen, Carlsbad, Calif.) or Cytofectin™ (Genlantis, San Diego, Calif.). Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art (Sambrook and Russell in Molecular Cloning. A Laboratory Manual. Third Edition. Cold Spring Harbor Laboratory Press, Cold Spring Harbor, N.Y. 2001). RNA is prepared using methods well known in the art, for example, using the TRIZOL® Reagent (Invitrogen, Carlsbad, Calif.) according to the manufacturer's recommended protocols.

### Analysis of Inhibition of Target Levels or Expression

Inhibition of levels or expression of an ApoCIII nucleic acid can be assayed in a variety of ways known in the art (Sambrook and Russell in Molecular Cloning. A Laboratory Manual. Third Edition. Cold Spring Harbor Laboratory Press, Cold Spring Harbor, N.Y. 2001). For example, target nucleic acid levels can be quantitated by, e.g., Northern blot analysis, competitive polymerase chain reaction (PCR), or quantitative real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM® 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT and real-time-PCR reactions are carried out by methods well known to those skilled in the art.

Gene (or RNA) target quantities obtained by real time PCR can be normalized using either the expression level of a gene whose expression is constant, such as cyclophilin A, or by quantifying total RNA using RIBOGREEN® (Invitrogen, Inc. Carlsbad, Calif.). Cyclophilin A expression is quantified by real time PCR, by being run simultaneously with the target, multiplexing, or separately. Total RNA is quantified using RIBOGREEN® RNA quantification reagent (Invitrogen, Inc. Carlsbad, Calif.). Methods of RNA quantification by RIBOGREEN® are taught in Jones, L. J., et al, (Analytical Biochemistry, 1998, 265, 368-374). A CYTOFLUOR® 4000 instrument (PE Applied Biosystems, Foster City, Calif.) is used to measure RIBOGREEN® fluorescence.

Probes and primers are designed to hybridize to an ApoCIII nucleic acid. Methods for designing real-time PCR probes and primers are well known in the art, and may include the use of software such as PRIMER EXPRESS® Software (Applied Biosystems, Foster City, Calif.).

Gene target quantities obtained by RT, real-time PCR can use either the expression level of GAPDH or Cyclophilin A, genes whose expression are constant, or by quantifying total RNA using RiboGreen (Molecular Probes, Inc. Eugene, Oreg.). GAPDH or Cyclophilin A expression can be quantified by RT, real-time PCR, by being run simultaneously with the target, multiplexing, or separately. Total RNA was quantified using RiboGreen™ RNA quantification reagent (Molecular Probes, Inc. Eugene, Oreg.).

### Analysis of Protein Levels

Antisense inhibition of ApoCIII nucleic acids can be assessed by measuring ApoCIII protein levels. Protein levels of ApoCIII can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS) (Sambrook and Russell in Molecular Cloning. A Laboratory Manual. Third Edition. Cold Spring Harbor Laboratory Press, Cold Spring Harbor, N.Y. 2001). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Antibodies useful for the detection of human and mouse ApoCIII are commercially available.

### In Vivo Testing of Antisense Compounds

Antisense compounds, for example, antisense oligonucleotides, are tested in animals to assess their ability to inhibit expression of ApoCIII and produce phenotypic changes. Testing can be performed in normal animals, or in experimental disease models. For administration to animals, antisense oligonucleotides are formulated in a pharmaceutically acceptable diluent, such as phosphate-buffered saline. Administration includes parenteral routes of administration. Calculation of antisense oligonucleotide dosage and dosing frequency depends upon factors such as route of administration and animal body weight. Following a period of treatment with antisense oligonucleotides, RNA is isolated from tissue and changes in ApoCIII nucleic acid expression are measured. Changes in ApoCIII protein levels are also measured.

### Certain Indications

Novel effects of ApoCIII inhibition in patients with Lipodystrophy (General Lipodystrophy or Partial Lipodystrophy) have been identified and disclosed herein. The example disclosed hereinbelow disclose reductions in TG and increases in HDL among other biomarkers in Lipodystrophy patients.

In certain embodiments, provided herein are methods of treating a Lipodystrophy subject comprising administering one or more pharmaceutical compositions as described herein. In certain embodiments, the pharmaceutical composition comprises an antisense compound targeted to an ApoCIII.

In certain embodiments, administration of an antisense compound targeted to an ApoCIII nucleic acid to a subject with Lipodystrophy results in reduction of ApoCIII expression by at least about 15, 20, 25, 30, 35, 40, 45, 50, 55, 60, 65, 70, 75, 80, 85, 90, 95 or 99%, or a range defined by any two of these values. In certain embodiments, ApoCIII expression is reduced to ≤50 mg/L, ≤60 mg/L, ≤70 mg/L, ≤80 mg/L, ≤90 mg/L, ≤100 mg/L, ≤110 mg/L, ≤120 mg/L, ≤130 mg/L, ≤140 mg/L, ≤150 mg/L, ≤160 mg/L, ≤170 mg/L, ≤180 mg/L, ≤190 mg/L or ≤200 mg/L.

In certain embodiments, the subject has a disease or disorder related to Lipodystrophy. In certain embodiments, the subject has a disease or disorder related to Generalized Lipodystrophy. In certain embodiments, the subject has a disease or disorder related to Partial Lipodystrophy. In certain embodiments the disease or disorder is a cardiovascular or metabolic disease or disorder in a subject with Lipodystrophy. In certain embodiments, the cardiovascular disease or disorder includes, but is not limited to, aneurysm, angina, arrhythmia, atherosclerosis, cerebrovascular disease, coronary heart disease, hypertension, dyslipidemia, hyperlipidemia, hypertriglyceridemia, hypercholesterolemia, stroke and the like. In certain embodiments, the metabolic disease or disorder include, but is not limited to, hyperglycemia, prediabetes, diabetes (type I and type II), obesity, insulin resistance, metabolic syndrome and diabetic dyslipidemia. In certain embodiments, the disease or disorder is hypertriglyceridemia in a subject with Lipodystrophy. In certain embodiments, the disease or disorder is pancreatitis in a subject with Lipodystrophy. In certain embodiments, the disease or disorder is NAFLD or NASH in a subject with Lipodystrophy. In certain embodiments, the disease or disorder is cirrhosis or hepatocarcinoma in a subject with Lipodystrophy

In certain embodiments, compounds targeted to ApoCIII as described herein modulate physiological markers or phenotypes of pancreatitis, a cardiovascular or a metabolic disease or disorder in a subject with Lipodystrophy. In certain of the experiments, the compounds can increase or decrease physiological markers or phenotypes compared to untreated animals. In certain embodiments, the increase or decrease in physiological markers or phenotypes is associated with inhibition of ApoCIII by the compounds described herein.

In certain embodiments, physiological markers or phenotype of a cardiovascular disease or disorder can be quantifiable. For example, TG or HDL levels can be measured and quantified by, for example, standard lipid tests. In certain embodiments, physiological markers or phenotypes such as HDL can be increased by about 5, 10, 15, 20, 25, 30, 35, 40, 45, 50, 55, 60, 65, 70, 75, 80, 85, 90, 95 or 99%, or a range defined by any two of these values. In certain embodiments, physiological markers phenotypes such as TG (postprandial or fasting) can be decreased by about 5, 10, 15, 20, 25, 30, 35, 40, 45, 50, 55, 60, 65, 70, 75, 80, 85, 90, 95 or 99%, or a range defined by any two of these values. In certain embodiments, TG (postprandial or fasting) is reduced to ≤100 mg/dL, ≤110 mg/dL, ≤120 mg/dL, ≤130 mg/dL, ≤140 mg/dL, ≤150 mg/dL, ≤160 mg/dL, ≤170 mg/dL, ≤180 mg/dL, ≤190 mg/dL, ≤200 mg/dL, ≤210 mg/dL, ≤220 mg/dL, ≤230 mg/dL, ≤240 mg/dL, ≤250 mg/dL, ≤260 mg/dL, ≤270 mg/dL, ≤280 mg/dL, ≤290 mg/dL, ≤300 mg/dL, ≤350 mg/dL, ≤400 mg/dL, ≤450 mg/dL, ≤500 mg/dL, ≤550 mg/dL, ≤600 mg/dL, ≤650 mg/dL, ≤700 mg/dL, ≤750 mg/dL, ≤800 mg/dL, ≤850 mg/dL, ≤900 mg/dL, ≤950 mg/dL, ≤1000 mg/dL, ≤1100 mg/dL, ≤1200 mg/dL, ≤1300 mg/dL, ≤1400 mg/dL, ≤1500 mg/dL, ≤1600 mg/dL, ≤1700 mg/dL, ≤1800 mg/dL or ≤1900 mg/dL.

In certain embodiments, physiological markers or phenotypes of a metabolic disease or disorder can be quantifiable. For example, glucose levels or insulin resistance can be measured and quantified by standard tests known in the art. In certain embodiments, physiological markers or phenotypes such as glucose levels or insulin resistance can be decreased by about 5, 10, 15, 20, 25, 30, 35, 40, 45, 50, 55, 60, 65, 70, 75, 80, 85, 90, 95 or 99%, or a range defined by any two of these values. In certain embodiments, physiological markers phenotypes such as insulin sensitivity can be increased by about 5, 10, 15, 20, 25, 30, 35, 40, 45, 50, 55, 60, 65, 70, 75, 80, 85, 90, 95 or 99%, or a range defined by any two of these values.

Also, provided herein are methods for preventing, treating or ameliorating a symptom associated with a disease or disorder in a subject with Lipodystrophy with a compound described herein. In certain embodiments, provided is a method for reducing the rate of onset of a symptom associated or disease associated with Lipodystrophy. In certain embodiments, provided is a method for reducing the severity of a symptom or disease associated with Lipodystrophy. In such embodiments, the methods comprise administering to an individual with Lipodystrophy a therapeutically effective amount of a compound targeted to an ApoCIII nucleic acid. In certain embodiments the disease or disorder is pancreatitis or a cardiovascular or metabolic disease or disorder.

Cardiovascular diseases or disorders are characterized by numerous physical symptoms. Any symptom known to one of skill in the art to be associated with a cardiovascular disease can be prevented, treated, ameliorated or otherwise modulated as set forth in the methods described herein. In certain embodiments, the symptom can be any of, but not limited to, angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen or fever.

Metabolic diseases or disorders are characterized by numerous physical symptoms. Any symptom known to one of skill in the art to be associated with a metabolic disorder can be prevented, treated, ameliorated or otherwise modulated as set forth in the methods described herein. In certain embodiments, the symptom can be any of, but not limited to, excessive urine production (polyuria), excessive thirst and increased fluid intake (polydipsia), blurred vision, unexplained weight loss and lethargy.

Pancreatitis is characterized by numerous physical symptoms. Any symptom known to one of skill in the art to be associated with a pancreatitis can be prevented, treated, ameliorated or otherwise modulated as set forth in the methods described herein. In certain embodiments, the symptom can be any of, but not limited to, abdominal pain, vomiting, nausea, and abdominal sensitivity to pressure.

In certain embodiments, provided are methods of treating a subject with Lipodystrophy comprising administering a therapeutically effective amount of one or more pharmaceutical compositions as described herein. In certain embodiments, administration of a therapeutically effective amount of an antisense compound targeted to an ApoCIII nucleic acid is accompanied by monitoring of ApoCIII levels or disease markers associated with Lipodystrophy to determine a subject's response to the antisense compound. A subject's response to administration of the antisense compound is used by a physician to determine the amount and duration of therapeutic intervention.

In certain embodiments, pharmaceutical compositions comprising an antisense compound targeted to ApoCIII are used for the preparation of a medicament for treating a subject with Lipodystrophy.

### Administration

The compounds or pharmaceutical compositions of the present invention can be administered in a number of ways depending upon whether local or systemic treatment is desired and upon the area to be treated. Administration can be oral or parenteral.

In certain embodiments, the compounds and compositions as described herein are administered parenterally. Parenteral administration includes intravenous, intra-arterial, subcutaneous, intraperitoneal or intramuscular injection or infusion.

In certain embodiments, parenteral administration is by infusion. Infusion can be chronic or continuous or short or intermittent. In certain embodiments, infused pharmaceutical agents are delivered with a pump. In certain embodiments, the infusion is intravenous.

In certain embodiments, parenteral administration is by injection. The injection can be delivered with a syringe or a pump. In certain embodiments, the injection is a bolus injection. In certain embodiments, the injection is administered directly to a tissue or organ. In certain embodiments, parenteral administration is subcutaneous.

In certain embodiments, formulations for parenteral administration can include sterile aqueous solutions which can also contain buffers, diluents and other suitable additives such as, but not limited to, penetration enhancers, carrier compounds and other pharmaceutically acceptable carriers or excipients.

In certain embodiments, formulations for oral administration of the compounds or compositions of the invention can include, but is not limited to, pharmaceutical carriers, excipients, powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In certain embodiments, oral formulations are those in which compounds of the invention are administered in conjunction with one or more penetration enhancers, surfactants and chelators.

### Dosing

In certain embodiments, pharmaceutical compositions are administered according to a dosing regimen (e.g., dose, dose frequency, and duration) wherein the dosing regimen can be selected to achieve a desired effect. The desired effect can be, for example, reduction of ApoCIII or the prevention, reduction, amelioration or slowing the progression of a disease or condition associated with Lipodystrophy.

In certain embodiments, the variables of the dosing regimen are adjusted to result in a desired concentration of pharmaceutical composition in a subject. “Concentration of pharmaceutical composition” as used with regard to dose regimen can refer to the compound, oligonucleotide, or active ingredient of the pharmaceutical composition. For example, in certain embodiments, dose and dose frequency are adjusted to provide a tissue concentration or plasma concentration of a pharmaceutical composition at an amount sufficient to achieve a desired effect.

Dosing is dependent on severity and responsiveness of the disease state to be treated, with the course of treatment lasting from several days to several months, or until a cure is effected or a diminution of the disease state is achieved. Dosing is also dependent on drug potency and metabolism. In certain embodiments, dosage is from 0.01 μg to 100 mg per kg of body weight, or within a range of 0.001 mg-1000 mg dosing, and may be given once or more daily, weekly, monthly or yearly, or even once every 2 to 20 years. Following successful treatment, it may be desirable to have the patient undergo maintenance therapy to prevent the recurrence of the disease state, wherein the oligonucleotide is administered in maintenance doses, ranging from 0.01 μg to 100 mg per kg of body weight, once or more daily, to once every 20 years or ranging from 0.001 mg to 1000 mg dosing.

### Certain Combination Therapies

In certain embodiments, a first agent comprising the compound described herein is co-administered with one or more secondary agents. In certain embodiments, such second agents are designed to treat the same disease, disorder, or condition as the first agent described herein. In certain embodiments, such second agents are designed to treat a different disease, disorder, or condition as the first agent described herein. In certain embodiments, a first agent is designed to treat an undesired side effect of a second agent. In certain embodiments, second agents are co-administered with the first agent to treat an undesired effect of the first agent. In certain embodiments, such second agents are designed to treat an undesired side effect of one or more pharmaceutical compositions as described herein. In certain embodiments, second agents are co-administered with the first agent to produce a combinational effect. In certain embodiments, second agents are co-administered with the first agent to produce a synergistic effect. In certain embodiments, the co-administration of the first and second agents permits use of lower dosages than would be required to achieve a therapeutic or prophylactic effect if the agents were administered as independent therapy. In certain embodiments, the first agent is administered to a subject that has failed or become non-responsive to a second agent. In certain embodiments, the first agent is administered to a subject in replacement of a second agent.

In certain embodiments, one or more compositions described herein and one or more other pharmaceutical agents are administered at the same time. In certain embodiments, one or more compositions of the invention and one or more other pharmaceutical agents are administered at different times. In certain embodiments, one or more compositions described herein and one or more other pharmaceutical agents are prepared together in a single formulation. In certain embodiments, one or more compositions described herein and one or more other pharmaceutical agents are prepared separately.

In certain embodiments, second agents include, but are not limited to, growth hormone-releasing factor (GRF), leptin replacement agent, ApoCIII lowering agent, DGAT1 inhibitor, cholesterol lowering agent, non-HDL lipid lowering (e.g., LDL) agent, HDL raising agent, fish oil, niacin (nicotinic acid), fibrate, statin, DCCR (salt of diazoxide), glucose-lowering agent and/or anti-diabetic agents. In certain embodiments, the first agent is administered in combination with the maximally tolerated dose of the second agent. In certain embodiments, the first agent is administered to a subject that fails to respond to a maximally tolerated dose of the second agent.

An example of a leptin replacement agent is Myalept®.

An example of a growth hormone-releasing factor (GRF) is Egrifta®.

Examples of ApoCIII lowering agents include an ApoCIII antisense oligonucleotide different from the first agent, fibrate or an Apo B antisense oligonucleotide.

An example of a DGAT1 inhibitor is LCQ908 (Novartis Pharmaceuticals).

Examples of glucose-lowering and/or anti-diabetic agents include, but is not limited to, a therapeutic lifestyle change, PPAR agonist, a dipeptidyl peptidase (IV) inhibitor, a GLP-1 analog, insulin or an insulin analog, an insulin secretagogue, a SGLT2 inhibitor, a human amylin analog, a biguanide, an alpha-glucosidase inhibitor, metformin, sulfonylurea, rosiglitazone, meglitinide, thiazolidinedione, alpha-glucosidase inhibitor and the like. The sulfonylurea can be acetohexamide, chlorpropamide, tolbutamide, tolazamide, glimepiride, a glipizide, a glyburide, or a gliclazide. The meglitinide can be nateglinide or repaglinide. The thiazolidinedione can be pioglitazone or rosiglitazone. The alpha-glucosidase can be acarbose or miglitol.

The cholesterol or lipid lowering therapy can include, but is not limited to, a therapeutic lifestyle change, statins, bile acids sequestrants, nicotinic acid and fibrates. The statins can be atorvastatin, fluvastatin, lovastatin, pravastatin, rosuvastatin and simvastatin and the like. The bile acid sequestrants can be colesevelam, cholestyramine, colestipol and the like. The fibrates can be gemfibrozil, fenofibrate, clofibrate and the like. The therapeutic lifestyle change can be dietary fat restriction.

HDL increasing agents include cholesteryl ester transfer protein (CETP) inhibiting drugs (such as Torcetrapib), peroxisome proliferation activated receptor agonists, Apo-A1, Pioglitazone and the like.

### Certain Treatment Populations

In certain embodiments, the compounds, compositions and methods described herein are useful in treating subjects with Lipodystrophy. Subjects with Lipodystrophy are at a significant risk of pancreatitis, cardiovascular and metabolic disease. For these subjects, recurrent pancreatitis is a debilitating and potentially lethal complication; other clinical sequelae include increased tendency for atherosclerosis and diabetes.

Lipodystrophy syndromes are a group of rare metabolic diseases characterized by selective loss of adipose tissue that leads to ectopic fat deposition in liver and muscle and the development of insulin resistance, diabetes, dyslipidemia and fatty liver disease. These syndromes are classified according to the underlying etiology (inherited or acquired) and according to the distribution of fat loss into Generalized or Partial Lipodystrophies (Garg et al., J Clin Endocrinol Metab, 2011, 96: 3313-3325; Chan et al., Endocr Pract, 2010, 16: 310-323; Simha et al., Curr Opin Lipidol, 2006, 17(2): 162-169; Garg, N Engl J Med, 2004, 350: 1220-1234).

A. Generalized Lipodystrophy

Generalized Lipodystrophy has a prevalence of 1 in 1 million (Garg et al., J Clin Endocrinol Metab, 2011, 96: 3313-3325). Congenital generalized lipodystrophy (CGL) is the main subtype of inherited lipodystrophy with a prevalence of 1 in 10 million (National Organization for Rare Disorders [NORD], The Physician's Guide to Lipodystrophy Disorders, 2012). The diagnosis of CGL is usually made at birth and approximately 300 cases have been reported. Acquired generalized lipodystrophy usually presents in childhood or adolescence and has been reported in approximately 100 cases. The exact mechanism of fat loss is not known: 50% are idiopathic, 25% are preceded by panniculitis and 25% have associated autoimmune diseases (ie. juvenile dermatomyositis). The clinical phenotype of Generalized Lipodystrophies includes total loss of subcutaneous and visceral fat, low levels of leptin and adiponectin, hyperinsulinemia, diabetes, hypertriglyceridemia and nonalcoholic fatty liver disease (NAFLD). Cirrhosis is more common in CGL.

B. Partial Lipodystrophy

Partial Lipodystrophy is an ultra-orphan indication for which there is a significant unmet medical need. Diabetes and/or hypertriglyceridemia associated with this condition can lead to serious complications (Handelsman et al., Endocrine Practice, 2013; 19 (1): 107-116): acute pancreatitis, especially when triglyceride levels are >1000 mg/dL; accelerated microvascular complications from uncontrolled diabetes; accelerated cardiovascular disease from lipid abnormalities and insulin resistance; steatohepatitis that can progress to cirrhosis; and/or proteinuric nephropathies which can progress to end stage renal disease.

Partial Lipodystrophy may have a higher prevalence than generalized lipodystrophy, but the true prevalence is unknown as these patients are greatly under-diagnosed (Garg et al., J Clin Endocrinol Metab, 2011, 96: 3313-3325; Chan et al., Endocr Pract, 2010, 16: 310-323).

Both genetic and acquired forms exist for Partial Lipodystrophy. Acquired lipodystrophies are caused by medications, autoimmune mechanisms or other unknown mechanisms (idiopathic). An acquired form seen in patients with the human immunodeficiency virus (HIV) on protease inhibitors has become the most prevalent form of Partial Lipodystrophy, with an estimate of 100,000 patients in the United States and many more in other countries.

Acquired Partial Lipodystrophy (APL, Barraquer-Simons syndrome) has been reported in approximately 250 cases. The onset of the disease usually occurs before age 15. Patients lose subcutaneous fat gradually starting at the face and spreading downward and most patients present with fat loss from the face, neck, upper extremities and trunk, with sparing of abdomen and lower extremities. The loss of adipose tissue is probably autoimmune-mediated as evidenced by low serum levels of complement 3 and complement 3-nephritic factor. Metabolic complications are rare but one fifth of patients develop membranoproliferative glomerulonephritis.

Familial Partial Lipodystrophy (FPL), described in the 1970s independently by Kobberling and Dunnigan, is the most common subtype of inherited partial lipodystrophy (National Organization for Rare Disorders [NORD], The Physician's Guide to Lipodystrophy Disorders, 2012). FPL encompasses several subtypes differentiated by the underlying genetic mutation (Six FPL subtypes and mutations in 5 genes have been identified). FPL type 1, Kobberling variety, has been reported in a handful of individuals and its molecular basis is unknown. FPL type 2, Dunnigan variety, is the most common form and the most well characterized disorder and is due to missense mutations in the A and C LMNA gene. FPL type 3 has been reported in 30 patients and is due to mutations in the PPARγ gene. FPL type 4 has been reported in 5 patients and is due to mutations in the PLIN1 gene. FPL type 5 has been reported in 4 members of a family who presented with insulin resistance and diabetes and is due to mutations in the AKT2 gene. The last subtype, Autosomal Recessive FPL, has been identified recently in one patient with homozygous mutation in CIDEC. Some individuals with FPL do not have mutations in any of these genes, suggesting that additional, as yet unidentified genes can cause the disorder.

The diagnosis of Partial Lipodystrophy is mainly clinical and needs to be considered in patients presenting with the triad of insulin resistance (with or without overt diabetes), significant dyslipidemia in the form of hypertriglyceridemia, and fatty liver (Huang-Dorang et al., J Endocrinol, 2010, 207: 245-255). Patients often present with diabetes and severe insulin resistance requiring high doses of insulin. Other evidence of severe insulin resistance is provided by the presence of acanthosis nigricans and polycystic ovarian syndrome (with symptoms like hyperandrogenism and oligomenorrhea). Some patients develop severe hypertriglyceridemia resulting in episodes of pancreatitis. In many patients, the triglyceride (TG) levels remain persistently elevated despite fully optimized therapy or diet modifications. Radiographic evidence of hepatic steatosis or steatohepatitis with hepatomegaly and/or elevated transaminases is not unusual (Handelsman et al., Endocrine Practice, 2013, 19 (1): 107-116). Compared to the other subtypes, the FPL type 3 seems to have milder metabolic abnormalities. These patients may also have abnormal LH/FSH secretion and fertility problems, as well as cardiovascular and kidney pathology (Handelsman et al., Endocrine Practice, 2013, 19 (1): 107-116). Patients with the Dunnigan variety have a higher risk of coronary artery disease and other types of atherosclerotic vascular disease. Although very rare, patients with a specific mutation in the LMNA gene are at an increased risk of cardiomyopathy and its associated complications, congestive heart failure and conduction defects.

Careful clinical assessment of fat distribution through visual and physical examination can confirm the diagnosis. Patients with FPL have reduced subcutaneous fat in the limbs and truncal regions and may have excess subcutaneous fat deposition in neck, face and intraabdominal regions. Patients with the Dunnigan variety have normal body fat distribution in childhood and gradually lose subcutaneous fat from the extremities and trunk around the time of puberty. In women, the loss of fat may be most striking in the buttocks and hips. At the same time these patients accumulate fat on the face (“double chin”) and neck and upper back (“Cushingoid appearance with buffalo hump”). In the Kobberling variety, fat loss is generally confined to the arms and legs. In patients with PPAR gamma mutations, the fast loss has a more distal distribution being more prominent in calf and forearm than in thighs and arms. In patients with the PLIN1 mutations the fat loss was more prominent in the lower limbs and buttocks. In patients with the AKT2 mutations the fat loss is more prominent in arms and legs. The extent of adipose tissue loss usually determines the severity of the metabolic abnormalities. Patients display prominent muscularity and phlebomegaly (enlarged veins) in the extremities and complain of disproportionate hyperphagia. The condition in females is more easily recognized than in men, and so is reported more often. Patients may also have a family history of similar physical appearance and/or fat loss.

Genetic testing, when available, is confirmatory. (Hegele et al., J. Lipid Res, 2007, 48: 1433-1444; Garg et al., J Clin Endocrinol Metab, 2011, 96: 3313-3325; Huang-Dorang et al., J Endocrinol, 2010, 207: 245-255).

C. Currently Available Treatments for Lipodystrophy

Current treatment for Lipodystrophies includes lifestyle modification reducing caloric intake and increasing energy expenditure via exercise. Conventional therapies used to treat severe insulin resistance (e.g., metformin, thiazolidinediones, GLP-1s, insulin), and/or high TGs (e.g., fibrates, fish oils) are not very efficacious in these patients (Chan et al., Endocr Pract, 2010, 16: 310-323).

In patients with HIV-associated Lipodystrophy, Egrifta® (tesamorelin) is commercially available to reduce excess abdominal fat (Egrifta® Package Insert, 2013). Egrifta®, a growth hormone releasing factor, was evaluated in two clinical trials involving 816 HIV-infected adult men and women with lipodystrophy and excess abdominal fat. Egrifta® showed greater reductions in abdominal fat as measured by CT scan compared to placebo. Some patients reported improvements in their self-image (Egrifta® Package Insert, 2013).

In patients with Generalized Lipodystrophy, metabolic complications are related to leptin deficiency. Myalept® (metreleptin) has been approved as leptin replacement therapy to treat the complications of leptin deficiency in addition to diet in patients with congenital or acquired Generalized Lipodystrophy (Myalept® Package Insert, 2014). The safety and effectiveness of Myalept® was evaluated in two open-label studies conducted at the NIH which included 72 patients (48 with generalized lipodystrophy and 24 with partial lipodystrophy) with diabetes, high TG, and elevated levels of fasting insulin. Myalept® was effective at reducing HbA1c, fasting glucose, and triglycerides (Myalept®, FDA Briefing Document, 2013; Oral et al., N Engl J Med, 2002, 346: 570-578; Chan et al., Endocr Pract, 2011, 17(6): 922-932).

In patients with Partial Lipodystrophy, Myalept® had a more varied and attenuated response in the NIH conducted clinical trial. While all patients with Generalized Lipodystrophy had low leptin levels [mean (SD): 1.3 (1.1) ng/mL], patients with Partial Lipodystrophy had a wider range of baseline leptin values [mean (SD): 4.9 (3.1) ng/mL]. In patients with Partial Lipodystrophy, greater improvement in metabolic variables was observed for patients with low baseline leptin concentration. For example, while the average change from baseline in HbA1c at month 12 was −0.9% for patients with Partial Lipodystrophy and low leptin levels, it was only −0.1% for those with Partial Lipodystrophy and higher leptin levels (Myalept®, FDA Briefing Document, 2013).

Due to safety concerns, Myalept® is available only through a risk evaluation and mitigation strategy (REMS) program, which requires prescriber and pharmacy certification and special documentation (Myalept®, FDA Briefing Document, 2013; Chan et al., Endocr Pract, 2011, 17(6): 922-932). Three cases of T-cell lymphoma have been reported in patients with acquired Generalized Lipodystrophy taking Myalept®. The majority of patients exposed to Myalept® treatment developed anti-drug antibodies with neutralizing activity to endogenous leptin or Myalept®; this may potentially lead to severe infections or loss of treatment effectiveness.

No specific pharmacologic treatment currently exists for non-iatrogenic forms of Partial Lipodystrophy.

Accordingly, there is a need to provide patients with Lipodystrophy novel treatment options.

ApoCIII inhibition is known to decrease TG levels, decrease HbA1c levels and/or raises HDL levels in subjects. Reducing TG, HbA1c and/or raising HDL levels, ApoCIII inhibition with the compounds and compositions described herein may prevent, treat, delay or ameliorate Lipodystrophy, or symptom thereof, in a patient. Reducing TG, HbA1c and/or raising HDL levels, ApoCIII inhibition with the compounds and compositions described herein may prevent, treat, delay or ameliorate a disease, disorder, or symptom thereof, associated with Lipodystrophy. ApoCIII inhibition with the compounds and compositions described herein may prevent, treat, delay, ameliorate or reduce the risk of cardiovascular disease in patients with Lipodystrophy. ApoCIII inhibition with the compounds and compositions described herein may prevent, treat, delay, ameliorate or reduce the risk of metabolic disease in patients with Lipodystrophy. ApoCIII inhibition with the compounds and compositions described herein may prevent, treat, delay, ameliorate or reduce the risk of pancreatitis in patients with Lipodystrophy. ApoCIII inhibition with the compounds and compositions described herein may improve the metabolic profile of patients with Lipodystrophy. ApoCIII inhibition with the compounds and compositions described herein may prevent, treat, delay, ameliorate or reduce the number and/or severity of complications associated with diabetes in patients with Lipodystrophy. ApoCIII inhibition with the compounds and compositions described herein may prevent, treat, delay, ameliorate or reduce the number and/or severity of complications associated with diabetes in patients with Lipodystrophy. ApoCIII inhibition with the compounds and compositions described herein may improve insulin sensitivity in patients with Lipodystrophy. ApoCIII inhibition with the compounds and compositions described herein may prevent, treat, delay, ameliorate or reduce hepatic steatosis, NAFLD, NASH and/or liver cirrhosis in patients with Lipodystrophy,

### Certain Compounds

We have previously disclosed compositions comprising antisense compounds targeting ApoCIII and methods for inhibiting ApoCIII by the antisense compounds in US 20040208856 (U.S. Pat. No. 7,598,227), US 20060264395 (U.S. Pat. No. 7,750,141), WO 2004/093783 and WO 2012/149495, all incorporated-by-reference herein. In these applications, a series of antisense compounds was designed to target different regions of the human ApoCIII RNA, using published sequences (nucleotides 6238608 to 6242565 of GenBank accession number NT_035088.1, representing a genomic sequence, incorporated herein as SEQ ID NO: 4, and GenBank accession number NM_000040.1, incorporated herein as SEQ ID NO: 1). The compounds were chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-O-(2-methoxyethyl) nucleotides, also known as (2′-MOE) nucleotides. The internucleoside (backbone) linkages are phosphorothioate (P═S) throughout the oligonucleotide. All cytosine residues are 5-methylcytosines.

The antisense compounds were analyzed for their effect on human ApoCIII mRNA levels in HepG2 cells by quantitative real-time PCR. Several compounds demonstrated at least 45% inhibition of ApoCIII mRNA and are therefore preferred. Several compounds demonstrated at least 50% inhibition of human ApoCIII mRNA and are therefore preferred. Several compounds demonstrated at least 60% inhibition of human ApoCIII mRNA and are therefore preferred. Several compounds demonstrated at least 70% inhibition of human ApoCIII mRNA and are therefore preferred. Several compounds demonstrated at least 80% inhibition of human ApoCIII mRNA and are therefore preferred. Several compounds demonstrated at least 90% inhibition of human ApoCIII mRNA and are therefore preferred.

The target regions to which these preferred antisense compounds are complementary are referred to as “preferred target segments” and are therefore preferred for targeting by antisense compounds.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: ISIS 304801 Partial Lipodystrophy Clinical Trial

As described herein, a multi-center, randomized, double-blind, placebo-controlled study will be performed on patients with Partial Lipodystrophy to evaluate the response to, and the pharmacodynamic effects of, the Study Drug ISIS 304801. Patients with Partial Lipodystrophy have diabetes and other metabolic abnormalities, including elevated triglycerides, which increases their risk of pancreatitis. ISIS 304801 was previously disclosed in U.S. Pat. No. 7,598,227 and has the sequence 5′-AGCTTCTTGTCCAGCTTTAT-3′ (SEQ ID NO: 3) starting at position 508 on SEQ ID NO: 1 (GENBANK Accession No. NM_000040.1) or starting at position 3139 on SEQ ID NO: 2 (GENBANK Accession NT_033899.8 truncated from nucleotides 20262640 to 20266603). ISIS 304801 has a 5-10-5 MOE gapmer motif comprising a gap segment consisting of 10 linked deoxynucleosides, a 5′ wing segment consisting of 5 linked nucleosides, a 3′ wing segment consisting 5 linked nucleosides, wherein the gap segment is positioned immediately adjacent to and between the 5′ wing segment and the 3′ wing segment, wherein each nucleoside of each wing segment comprises a 2′-O-methyoxyethyl sugar, wherein each cytosine is a 5′-methylcytosine, and wherein each internucleoside linkage is a phosphorothioate linkage. ISIS 304801 has been shown to be potent in inhibiting ApoC-III and tolerable when administered to subjects.

### Patient Population

Up to 60 eligible patients meeting the following criteria will be enrolled in this clinical study.
A. Clinical diagnosis of lipodystrophy based on deficiency of subcutaneous body fat in a partial fashion assessed by physical examination, and at least 1 MAJOR criterion and 1 MINOR criterion (below):
   MAJOR Criteria
      a) Low skinfold thickness in anterior thigh by caliper measurement: men (≤10 mm) and women (≤22 mm) OR
      b) Genetic diagnosis of familial PL (e.g., mutations in LMNA, PPAR-γ, AKT2, CIDEC or PLIN1 genes)
   MINOR Criteria
      a) Insulin resistance defined as fasting insulin ≥20 mcU/mL
      b) Diabetes mellitus
      c) Acanthosis nigricans
      d) Polycystic Ovary Syndrome (PCOS) or PCOS-like symptoms (hirsutism, oligomenorrhea, and/or polycystic ovaries)
      e) History of pancreatitis associated with hypertriglyceridemia
      f) History of hepatic steatosis or steatohepatitis
      g) Similar fat distribution and/or history of fat loss in a first degree relative
      h) Prominent muscularity and phlebomegaly (enlarged veins) in the extremities
      i) Disproportionate hyperphagia
B. Fasting TG levels ≥500 mg/dL (≥5.7 mmol/L) at Screening and Baseline visit. If the fasting TG value at Screening and/or Baseline visit is <500 mg/dL (<5.7 mmol/L) but ≥350 mg/dL (≥4.0 mmol/L) up to two additional tests may be performed in order to qualify.

### Study Design

The patients will be randomized 1:1 (ISIS 304801: placebo) and stratified by ALT level (>2×upper limit of normal [ULN] vs. ≤2×ULN). For each patient the participation period consists of a ≤8-week screening period, which includes a ˜6-week diet stabilization period during which the patients will be encouraged to continue on their current diet. The baseline assessments will be performed at Week −2 to −1 during the screening period, and on Study Day 1 (first dose of drug administered to patient).

Following the diet stabilization, up to 60 eligible patients will be randomized 1:1 to receive ISIS 304801 300 mg or placebo once weekly for 52 weeks. Patients will be educated on self administration of drug. During the treatment period, patients will report to the study center for clinic visits a minimum of 10 times during Weeks 1-52 (around or in weeks 1, 4, 8, 12, 13, 19, 25, 26, 32, 38, 44, 51 and 52). Study Drug will be administered once weekly. Collection and measurement of vital signs, physical examination results, waist circumference, skinfold measurements, DEXA scans, electrocardiograms (ECGs), liver MRIs, echocardiograms, clinical laboratory parameters (including hematology; serum chemistry; lipid panel; plasma glucose, insulin, C-peptide, and CRP; urinalysis, and other analytes), ISIS 304801 plasma trough concentrations, immunogenicity testing, 7-point SMBG, collection of SMBG and hunger diary results, AEs, concomitant medication/procedure information, and quality of life assessments will be performed according to a schedule of procedures. Adverse Events (AEs) at the injection site should be collected as AEs. Dietary/alcohol counseling will commence at the start of the diet stabilization period and will be reinforced at intervals throughout the treatment and follow-up period.

Patients will be fasted prior to drawing all lipid samples and samples drawn locally must be sent to the central laboratory for analysis. Blood sampling for lipid panels at Weeks 12, 25 and 51 may be conducted by a home healthcare nurse if more convenient for the patient. Every effort should be made to ensure the previous week's dose is given 7 days prior to a scheduled clinic visit. Dosing instructions and training will be provided to the patient where applicable.

All visits will have a visit window of at least ±2 days. All reasonable attempts should be made to ensure compliance with the visit schedule. However, in the event that a visit does not occur or is delayed, all subsequent visits will be calculated based on the time elapsed since Day 1 rather than from the date of the previous visit.

After completion of the Week 52 visit assessments, patients will enter a 13-week post-treatment evaluation period. This period consists of two Study Center visits on Weeks 58 and 65. Alternatively, after completion of the Week 52 visit assessments, eligible patients may elect to receive ISIS 304801 in an open label extension (OLE) study, pending study approval by the IRB/IEC and the appropriate regulatory authority. In this case, patients will not participate in the post-treatment evaluation period.

Concomitant medications and AEs will be recorded throughout all periods of the study.

### Study Drug

A solution of the Study Drug ISIS 304801 (200 mg/mL, 1.5 mL) contained in prefilled syringes (PFS) will be provided. A trained professional will administer, or the patient will self-administer, 300 mg of the Study Drug as a single SC injection in the abdomen, thigh, or outer area of the upper arm on each dosing day.

### Results

A primary efficacy analysis will be performed to compare the percent change from baseline to the primary analysis time point in fasting TG between the ISIS 304801 treated and placebo groups in the Full Analysis Set (FAS). The primary efficacy analysis will take place after the last patient has completed the Week 52 visit and the database has been locked, and will be based on the percent change from baseline in fasting TG at the primary analysis time point (end of Month 3).

Secondary endpoints to be analyzed include: absolute change in fasting TG at 3, 6 and 12 months; proportion of patients who achieve a ≥40% reduction in fasting TG at 3, 6 and 12 months; change in HbA1c at 6, 9 and 12 months; change in fasting plasma glucose at 6, 9 and 12 months; and, change in liver volume and hepatic steatosis (as assessed by MRI) at 6 and 12 months.

Tertiary/Exploratory endpoints that may be assessed include the following:

Glycemic
Percent patients with HbA1c<7%
Percent of patients with HbA1c reduction >1% from baseline
Change in 24-hr glucose (using 7-point SMBG)
Change in HOMA-IR
Change in fasting insulin and C-peptide
Reduction in insulin use

Lipids
Change in other fasting lipid measurements: HDL-C, LDL-C, Total Cholesterol, VLDL-C, non-HDL-C, apoB (e.g., apoB-48 or apoB-100), apoA1, apoC-III (total, chylomicron, VLDL, LDL and HDL) and Free Fatty Acids (FFA)
Change in lipoprotein particle size/number

Adipose Tissue
Change in skinfold thickness and DEXA
Change in abdominal VAT and SAT volumes
Change in adiponectin and leptin
Change in body weight and waist circumference

Patient Reported Outcomes
Change in Quality of Life (EQ-5D, SF36)
Change in hunger scale
Change in widespread pain

Other
Change in testosterone

Results will be published when available.

### Pharmacokinetic (PK), Pharmacodynamic (PD) and Immunogenicity (IM) Analysis

Pharmacokinetic (PK), pharmacodynamic (PD) and immunogenicity properties of ISIS 304801 will be assessed and published when available.

### Safety Assessment

Safety endpoints to be assessed or methods of safety assessments include the following:
AEs including adjudicated events of pancreatitis and MACE
Vital signs and weight
Physical examinations
Clinical laboratory tests (serum chemistry, hematology, coagulation, urinalysis)
Echocardiography
ECGs
Use of concomitant medications
MRIs

Safety assessments will be published when available.

What is claimed is:

1. A method of treating, preventing, delaying or ameliorating Lipodystrophy in an animal comprising administering a therapeutically effective amount of a compound comprising an ApoCIII specific inhibitor to the animal, thereby preventing, delaying or ameliorating Lipodystrophy in the animal.

2. The method of claim 1, wherein administration of the compound reduces triglyceride levels in the animal.

3. The method of claim 1, wherein a symptom or risk of pancreatitis, a cardiovascular and/or metabolic disease or disorder is improved.

4. A method of reducing triglyceride levels in an animal with Lipodystrophy comprising administering a therapeutically effective amount of a compound comprising an ApoCIII specific inhibitor to the animal, whereby triglyceride levels are reduced in the animal with Lipodystrophy.

5. A method of preventing, delaying or ameliorating a cardiovascular and/or metabolic disease, disorder, condition, or symptom thereof, in an animal with Lipodystrophy comprising administering a therapeutically effective amount of a compound comprising an ApoCIII specific inhibitor to the animal, whereby the cardiovascular and/or metabolic disease, disorder, condition, or symptom thereof, is prevented, delayed or ameliorated in the animal with Lipodystrophy.

6. A method of preventing, delaying or ameliorating pancreatitis, or symptom thereof, in an animal with Lipodystrophy comprising administering a therapeutically effective amount of a compound comprising an ApoCIII specific inhibitor to the animal, whereby pancreatitis, or symptom thereof, is prevented, delayed or ameliorated in the animal with Lipodystrophy.

7. The method of claim 1, 4, 5 or 6, wherein the Lipodystrophy is Partial Lipodystrophy

8. The method of any preceding claim, wherein ApoCIII has a nucleic acid sequence as shown in SEQ ID NO: 1, SEQ ID NO: 2 or SEQ ID NO: 4.

9. The method of any preceding claim, wherein the compound comprises a nucleic acid inhibitor of ApoCIII.

10. The method of any preceding claim, wherein the compound comprises a modified oligonucleotide targeting ApoCIII.

11. The method of claim 10, wherein the modified oligonucleotide has a nucleobase sequence comprising at least 8 contiguous nucleobases of a nucleobase sequence of SEQ ID NO: 3.

12. The method of claim 10, wherein the nucleobase sequence of the modified oligonucleotide is at least 80%, at least 90% or 100% complementary to a nucleobase sequence of SEQ ID NO: 1, SEQ ID NO: 2 or SEQ ID NO: 4.

13. The method of any of claims 10-12, wherein the modified oligonucleotide consists of a single-stranded modified oligonucleotide.

14. The method of any of claims 10-13, wherein the modified oligonucleotide consists of 12 to 30 linked nucleosides.

15. The method of claim 14, wherein the modified oligonucleotide consists of 20 linked nucleosides.

16. The method of any of claims 10-15, wherein the modified oligonucleotide comprises at least one modified internucleoside linkage, sugar moiety or nucleobase.

17. The method of claim 16, wherein the at least one modified internucleoside linkage of the modified oligonucleotide is a phosphorothioate internucleoside linkage, the at least one modified sugar is a bicyclic sugar or 2′-O-methyoxyethyl and the at least one modified nucleobase is a 5-methylcytosine.

18. The method of claim 10, wherein the modified oligonucleotide comprises:
(a) a gap segment consisting of linked deoxynucleosides;
(b) a 5′ wing segment consisting of linked nucleosides;
(c) a 3′ wing segment consisting linked nucleosides;
wherein the gap segment is positioned immediately adjacent to and between the 5′ wing segment and the 3′ wing segment and wherein each nucleoside of each wing segment comprises a modified sugar.

19. The method of claim 10, wherein the modified oligonucleotide comprises:
(a) a gap segment consisting of 10 linked deoxynucleosides;
(b) a 5′ wing segment consisting of 5 linked nucleosides;
(c) a 3′ wing segment consisting 5 linked nucleosides;
wherein the gap segment is positioned immediately adjacent to and between the 5′ wing segment and the 3′ wing segment and wherein each nucleoside of each wing segment comprises a 2′-O-methyoxyethyl sugar, wherein each cytosine is a 5′-methylcytosine, and wherein at least internucleoside linkage is a phosphorothioate linkage.

20. A method of treating, preventing, delaying or ameliorating Partial Lipodystrophy, or a disease associated with Partial Lipodystophy in an animal comprising administering to the animal a therapeutically effective amount of a compound comprising a modified oligonucleotide having the sequence of SEQ ID NO: 3 wherein the modified oligonucleotide comprises:
(a) a gap segment consisting of 10 linked deoxynucleosides;
(b) a 5′ wing segment consisting of 5 linked nucleosides;
(c) a 3′ wing segment consisting 5 linked nucleosides;
wherein the gap segment is positioned immediately adjacent to and between the 5′ wing segment and the 3′ wing segment, wherein each nucleoside of each wing segment comprises a 2′-O-methyoxyethyl sugar, wherein each cytosine is a 5′-methylcytosine, wherein at least internucleoside linkage is a phosphorothioate linkage, and wherein the Partial Lipodystrophy, or a disease associated with Partial Lipodystophy, is treated, prevented, delayed or ameliorated in the animal.

21. A method of reducing triglyceride levels in an animal with Partial Lipodystrophy comprising administering to the animal a therapeutically effective amount of a compound comprising a modified oligonucleotide having the sequence of SEQ ID NO: 3 wherein the modified oligonucleotide comprises:
(a) a gap segment consisting of 10 linked deoxynucleosides;
(b) a 5′ wing segment consisting of 5 linked nucleosides;
(c) a 3′ wing segment consisting 5 linked nucleosides;
wherein the gap segment is positioned immediately adjacent to and between the 5′ wing segment and the 3′ wing segment, wherein each nucleoside of each wing segment comprises a 2′-O-methyoxyethyl sugar, wherein each cytosine is a 5′-methylcytosine, wherein at least one internucleoside linkage is a phosphorothioate linkage, and wherein the modified oligonucleotide reduces triglyceride levels in the animal with Partial Lipodystrophy.

22. A method of preventing, delaying or ameliorating a cardiovascular and/or metabolic disease, disorder, condition, or symptom thereof, in an animal with Partial Lipodystrophy by administering to the animal a therapeutically effective amount of a compound comprising a modified oligonucleotide having the sequence of SEQ ID NO: 3 wherein the modified oligonucleotide comprises:
(a) a gap segment consisting of 10 linked deoxynucleosides;
(b) a 5′ wing segment consisting of 5 linked nucleosides;
(c) a 3′ wing segment consisting 5 linked nucleosides;
wherein the gap segment is positioned immediately adjacent to and between the 5′ wing segment and the 3′ wing segment, wherein each nucleoside of each wing segment comprises a 2′-O-methyoxyethyl sugar, wherein each cytosine is a 5′-methylcytosine, and wherein at least one internucleoside linkage is a phosphorothioate linkage, wherein the modified oligonucleotide prevents, delays, ameliorates or reduces the cardiovascular and/or metabolic disease, disorder, condition, or symptom thereof, in the animal with Partial Lipodystrophy.

23. A method of preventing, delaying or ameliorating pancreatitis or symptom thereof, in an animal with Partial Lipodystrophy by administering to the animal a therapeutically effective amount of a compound comprising a modified oligonucleotide having the sequence of SEQ ID NO: 3 wherein the modified oligonucleotide comprises:
(a) a gap segment consisting of 10 linked deoxynucleosides;
(a) a 5′ wing segment consisting of 5 linked nucleosides;
(b) a 3′ wing segment consisting 5 linked nucleosides;
wherein the gap segment is positioned immediately adjacent to and between the 5′ wing segment and the 3′ wing segment, wherein each nucleoside of each wing segment comprises a 2′-O-methyoxyethyl sugar, wherein each cytosine is a 5′-methylcytosine, wherein at least one internucleoside linkage is a phosphorothioate linkage, and wherein the modified oligonucleotide prevents, delays, ameliorates or reduces the pancreatitis or symptom thereof.

24. A compound comprising an ApoCIII specific inhibitor for use in:
a. treating, preventing, delaying or ameliorating Partial Lipodystrophy, or a disease associated with Partial Lipodystophy in an animal;
b. reducing triglyceride levels in an animal with Partial Lipodystrophy;
c. increasing HDL levels and/or improving the ratio of TG to HDL in an animal with Partial Lipodystrophy;
d. preventing, delaying or ameliorating a cardiovascular and/or metabolic disease, disorder, condition, or a symptom thereof, in an animal with Partial Lipodystrophy; and/or
e. preventing, delaying or ameliorating pancreatitis, or a symptom thereof, in an animal with Partial Lipodystrophy.

25. The method or use of any preceding claim, wherein the compound is parenterally administered.

26. The method or use of claim 25, wherein the parenteral administration is subcutaneous administration.

27. The method or use of any preceding claim, further comprising a second agent.

28. The method or use of claim 27, wherein the second agent is selected from a leptin replacement therapy, ApoCIII lowering agent, cholesterol lowering agent, non-HDL lipid lowering agent, LDL lowering agent, TG lowering agent, cholesterol lowering agent, HDL raising agent, fish oil, niacin, fibrate, statin, DCCR (salt of diazoxide), glucose-lowering agent or anti-diabetic agents.

29. The method or use of claim 27, wherein the second agent is administered concomitantly or sequentially with the compound.

30. The method or use of any preceding claim, wherein the compound is a salt form.

31. The method or use of any preceding claim, further comprising a pharmaceutically acceptable carrier or diluent.

32. The method or use of any preceding claim, wherein the compound is conjugated.

33. The method or use of any preceding claim, wherein the Partial Lipodystrophy patient is identified by genetic screening.
